SEQUENCE LISTING (11 sequences)

SEQ ID NO: 1 - DNA, 30 bp, Artificial sequence
  oligo nucleotides
gttttcgaagattgggtggcactgttgtaa

SEQ ID NO: 2 - DNA, 30 bp, Artificial sequence
  oligo nucleotides
cagggcctcgttatcaatggagtaggtttc

SEQ ID NO: 3 - DNA, 30 bp, Artificial sequence
  oligo nucleotides
ctttgtcgagctggtatttggagaacacgt

SEQ ID NO: 4 - DNA, 30 bp, Artificial sequence
  oligo nucleotides
gcttctccttaatgtcacgcacgatttccc

SEQ ID NO: 5 - DNA, 30 bp, Artificial sequence; identical to SEQ ID NO: 3
  oligo nucleotides

SEQ ID NO: 6 - DNA, 25 bp, Artificial sequence
  oligo nucleotides
tcgagctggtatttggagaacacgt

SEQ ID NO: 7 - DNA, 30 bp, Artificial sequence
  oligo nucleotides
acgtgttctccaaataccagctcgacaaag

SEQ ID NO: 8 - DNA, 30 bp, Artificial sequence
  oligo nucleotides
acgtattctccaaataccagctcgacaaag

SEQ ID NO: 9 - DNA, 30 bp, Artificial sequence
  oligo nucleotides
acgtcttctccaaataccagctcgacaaag

SEQ ID NO: 10 - DNA, 30 bp, Artificial sequence
  oligo nucleotides
acgttttctccaaataccagctcgacaaag

SEQ ID NO: 11 - DNA, 30 bp, Artificial sequence
  oligo nucleotides
acgtattctacaaataccagctcgacaaag

(19) United States
(12) Patent Application Publication
(10) Pub. No.: US 2018/0121599 A1
(43) Pub. Date: May 3, 2018

(54) METHODS AND SYSTEMS FOR DETECTING A NUCLEIC ACID IN A SAMPLE BY ANALYZING HYBRIDIZATION

(71) Applicant: VITO NV (VLAAMSE INSTELLING VOOR TECHNOLOGISCH ONDERZOEK), Mol (BE)

(72) Inventors: Jef HOOYBERGHS, Mol (BE); Enrico CARLON, Heverlee (BE)

(73) Assignee: VITO NV (VLAAMSE INSTELLING VOOR TECHNOLOGISCH ONDERZOEK), Mol (BE)

(21) Appl. No.: 15/724,479

(22) Filed: Oct. 4, 2017

Related U.S. Application Data
(63) Continuation-in-part of application No. 15/253,973, filed on Sep. 1, 2016.
Application No. 15/253,973 is a continuation of application No. 13/394,253, filed on Mar. 5, 2012, now abandoned.

(51) Int. Cl. G06F 19/20; C12Q 1/6837

(52) U.S. Cl. CPC G06F 19/20; C12Q 1/6837

(57) ABSTRACT

A method for determining in a sample solution the presence of a target nucleic acid of interest, where the target nucleic acid of interest differs from one or more other target nucleic acids by only one or two nucleotides. The method comprises providing a first plurality of different probes and a second plurality of different probes, performing hybridization, where the hybridization includes contacting the sample solution with each probe of the first and second plurality of probes, and detecting a hybridization signal for each probe with the sample solution, determining the hybridization free energy for the hybridization between each probe of the first and second plurality of probes and the target nucleic acid of interest, and determining the presence of the target nucleic acid of interest based on the relationship between the hybridization signals detected for the plurality of probes.

### CROSS REFERENCE OF APPLICATIONS

The present application is a Continuation-in-Part application of U.S. Continuation patent application Ser. No. 15/253,973 filed on Sep. 1, 2016, herein incorporated by reference, which is based on U.S. patent application Ser. No. 13/394,253 which is the national stage application of International Patent Application No. PCT/EP2009/061510 filed on Sep. 5, 2009.

The invention relates to the field of detecting a target nucleic acid based on hybridization, such as in a microarray analysis, more particularly based on the analysis of hybridization. The present invention relates to methods and systems for analysis of hybridization, e.g. hybridization between a nucleic acid strand in solution and a complementary strand linked to a solid surface such as hybridization in microarrays.

### BACKGROUND OF THE INVENTION

Microarrays, such as DNA microarrays, are widely used in the current research in molecular biology. The devices have several important applications as for example in gene expression profiling, in the detection of single nucleotide polymorphisms, in the analysis of copy number variations, etc.

Targeted resequencing of genomic nucleic acids is often applied in diagnostic or prognostic tests, wherein samples of mixed sequence variants, of which some are possibly present in minority, need to be analyzed. For instance, biopsies from cancer tissue usually contain a mixture of cancerous and non-cancerous cells, whereby typically only the detection of the cancerous cells is of interest. Also, in the case of viral infections, a virus population within a single infected individual can have a high genomic variability. For instance, in the case of HIV, several mutations can be present even in a small genomic window of 20-30 nucleotides. For diagnostic purposes, it is often needed to identify genomic subsets where crucial mutations are known to occur. Therefore, there is a need to distinguish the presence of a specific mutation or variation out of a set of different potential variant sequences in a sample, the specific mutation or variation possibly being present in low abundance (minority) and occurring amongst a majority of ‘wild-type’ sequences.

It is known to use hybridization for mutation detection, such as in microarrays.

What all DNA microarrays have in common is the basic underlying reaction of hybridization between a nucleic acid strand in solution (target) and a complementary strand linked covalently at a solid surface (probe).

Hybridization is characterized by a (sequence dependent) free energy difference ΔG which measures the binding affinity for the two strands to form a duplex. In current microarray experiments it is assumed that the hybridization reaction has reached equilibrium when the optical read-out is performed. According to equilibrium thermodynamics the intensity I from a microarray spot is given by:

$$I = A \cdot \frac{c \cdot {e}^{( - Δ G / RT )}}{1 + c \cdot {e}^{( - Δ G / RT )}} \qquad [ 1 ]$$

with T the experimental temperature, R the gas constant, c the concentration of the target in solution, A an amplification factor of the optical reading device, and ΔG the free energy difference between the bound probe-target state (double stranded) and its unbound state.

This theory assumes that the hybridization process is a two-state process: either hybridization has taken place (bound state) or it has not (unbound state). This approximation is expected to be good for short sequences.

In case c. e^{(-ΔG/RT)}<<1 the expression can be simplified to

$$\begin{matrix}I = A \cdot c \cdot {e}^{( - Δ G / RT )} \\ or & [ 2 ] \\ log ( I ) = cte + log ( c ) - \frac{Δ G}{RT} & [ 3 ]\end{matrix}$$

To exploit this theory in technical applications, and thus to use it for analysis purposes, it is of central importance to have good estimates of ΔG for a given probe and target sequence. In the past decades, the static and dynamic properties of hybridization between two floating strands have been discussed. Nearest neighbor models provide reasonable approximation of the free energy difference for strands hybridization in solution. Such models estimate the hybridization free energy as a sum of dinucleotide parameters. These parameters were fitted through a series of (labour intensive) experiments. The relationship between hybridization in solution and hybridization in DNA microarrays is nevertheless not clear yet. A better understanding of the molecular interactions may result in the possibility to turn microarrays into more precise tools.

An often reported disadvantage of the use of hybridization for detection is specificity: the possibility of cross-hybridization of non-perfectly matching sequences to a probe sequence complicates the data analysis. This is particularly an issue for sample solutions containing two or more variants of a given sequence. As cross-hybridization is typically viewed as a limiting factor in the accuracy of analysis of hybridization, efforts are often aimed at avoiding cross-hybridization, such as by introducing chemical agents into the nucleic acid probes.

Accordingly, there is a need in the art for improved hybridization-based methods, such as microarray-based methods for detecting nucleic acids, particularly for the analysis of samples containing different variants of a given nucleic acid sequence.

### SUMMARY OF THE INVENTION

It is an object of embodiments of the present invention to provide good methods for analyzing or assessing hybridization of targets.

More particularly, it is an object of embodiments of the present invention to provide improved methods and systems for detecting a nucleic acid in a sample by hybridization.

The inventors have surprisingly found that by accurately quantifying the probe-target affinities for cross-hybridization, analysis of hybridization can provide a powerful detection method or a powerful targeted resequencing method. More particularly, in contrast to existing hybridization-based detection methods, the methods of the present invention involve analyzing the hybridization between a probe and a target nucleic acid which have at least one non-matching nucleotide, more particularly by measuring the hybridization intensities from multiple probes which are not perfectly matching to the target sequence. The analysis relies on estimates or calculations of the hybridization free energies of mismatching probe-nucleic acid duplexes (cross-hybridization signals). The data are then checked against the isotherm expected from equilibrium thermodynamics, in particular equations [1], [2] and [3] above.

The detection methods based on hybridization according to the present invention have the advantage that they can be provided as a simple test that can be miniaturized to a fully automated lab-on-chip, which can be used as point-of-care test. In addition, a DNA microarray based detection method according to the present invention benefits from the high quality and reproducibility of these devices and allows to fully exploit the generated data, i.e. both perfect matching data and data from cross-hybridizing sequences. Indeed, it is an advantage of embodiments according to the present invention that reliable and accurate results can be obtained by analysis of hybridization, e.g. in microarrays, allowing a wide range of hybridization based microarray applications.

It is an advantage of embodiments according to the present invention that methods and systems are provided for analyzing hybridization of targets, allowing identification and/or quantification of targets or providing relevant information regarding their hybridization. It is an advantage of embodiments according to the present invention that methods and systems are provided for setting up hybridization experiments for analyzing samples.

It is an advantage of embodiments according to the present invention that these provide a better understanding of the physico-chemical properties involved in the free energy of the molecular interactions, and of the time scale and the dynamics of the hybridization process. Embodiments according to the present invention allow a better characterization of free energy, a better characterization of the dynamics involved and allow taking this into account when performing hybridization experiments.

It is an advantage of embodiments according to the present invention that more accurate quantification of interactions in microarrays, such as e.g. DNA microarrays, can be performed. It is an advantage of such embodiments that this may lead to a better understanding of the functioning of microarrays and of other techniques involving hybridization on a surface.

It is an advantage of embodiments according to the present invention that methods and systems are provided allowing hybridization experiments in microarrays which are reliable, cheap and quick experiments. The methods and systems therefore are especially suited for diagnostics and personalized medicine, although embodiments of the present invention are not limited thereto.

The above objective is accomplished by a method and system according to the present invention.

The present invention is particularly based on a method for analyzing hybridization between a target in a sample solution and a probe bound at a surface, the method comprising receiving hybridization signal or hybridization signal intensities for hybridization of the target with a plurality of different probes, the probes being selected so that a range of hybridization detection intensity results for the hybridization between the target and the probe is covered, and analyzing the intensity of the hybridization signals as function of hybridization free energy. It is an advantage of embodiments according to the present invention that methods and systems are obtained allowing good analysis of the hybridization results. It is an advantage of embodiments according to the present invention that reliable and accurate results can be obtained by analysis of hybridization, e.g. in microarrays, allowing a wide range of hybridization based microarray applications.

An object of the present application provides a method for determining in a sample solution the presence of a target nucleic acid of interest, wherein the target nucleic acid of interest differs from one or more other target nucleic acids by only one or two nucleotides, the method comprising the steps of:

(a) providing
a first plurality of different probes wherein each probe comprises a nucleic acid that is perfectly complementary to one nucleic acid of a set of target nucleic acids which differ from the target nucleic acid of interest in only one or two nucleotides; and
a second plurality of different probes, wherein each probe comprises a nucleic acid that is non-perfectly complementary to each of said set of target nucleic acids which differ from the target nucleic acid of interest in only one or two nucleotides,

(b) performing a hybridization experiment, said hybridization experiment comprising contacting the sample solution with each probe of said first and second plurality of probes, and measuring a hybridization signal for each probe with the sample solution;

(c) determining—the hybridization free energy (i) for the hybridization between each probe of said first and said second plurality of probes and the target nucleic acid of interest or (ii) for the hybridization between each probe of said first and said second plurality of probes and said target nucleic acid of interest, based on a model for estimating the hybridization free energy as a sum of dinucleotide parameters.

(d) determining the presence of said target nucleic acid of interest based on the relationship between said hybridization signals measured for said plurality of probes in step (b) and said hybridization free energy determined in step (c).

Thus, in step (d), the hybridization signal intensity data is analyzed as function of the hybridization free energy and preferably comprises analyzing the logarithm of the hybridization intensity as function of the hybridization free energy for a range of hybridization free energies. Particularly, the analysis of the logarithm of the hybridization signal intensity as function of hybridization free energy comprises determining whether one linear relationship or a deviation therefrom can be distinguished between parts of the logarithm of the intensity and the hybridization free energy.

In particular embodiments, step (d) comprises determining whether said relationship between said hybridization signals measured for said plurality of probes in step (b) and said estimated or determined hybridization free energy in step (c) corresponds to the logarithmic relationship:

I=A. c. e^{(-ΔG}/_{RT)} (i.e. equation [2]) or with the linear relationship between log(I) and −ΔG/RT, as represented in equation [3];

wherein I is the hybridization intensity, A is an amplification factor, c the concentration of the target in solution and ΔG is hybridization free energy, T the experimental temperature and R the gas constant; and

wherein deviating from said logarithmic or linear relationship indicates the absence of said target nucleic acid of interest in the sample; and wherein not deviating from said logarithmic or linear relationship indicates the presence and/or concentration of said target nucleic acid of interest in the sample.

In particular embodiments, in step (d) the relation between said set of hybridization intensity data as a function of ΔΔG is considered, wherein ΔΔG corresponds to the difference in hybridization free energy ΔG between the target nucleic acid of interest and a non-perfectly complementary probe and the hybridization free energy ΔG of the target nucleic acid of interest and its perfectly matching probe (PM). Indeed, this amounts to using ΔΔG=ΔG−ΔG(PM); therefore, for a PM hybridization ΔΔG=0. As the free energies are shifted by a constant value, the same functional relationship as Equation [2] and [3] holds also for ΔΔG. Thus, in certain embodiments, step (d) comprises the step of calculating said hybridization free energy difference, such as by subtracting from all hybridization free energies determined in step (c) the hybridization free energy between said one nucleic acid of the plurality of known possible target nucleic acids and its perfect match, thus obtaining a set of ΔΔG values.

Accordingly, in preferred embodiments, (d) comprises the steps

(d1) subtracting from all hybridization free energies determined in step (c) the hybridization free energy between said target nucleic acid of interest and its perfectly matching probe, thus obtaining a set of ΔΔG values;

(d2) comparing the relation of said set of hybridization intensity data as a function of the ΔΔG values with the logarithmic relationship

I=A. c. e^{(-ΔΔG}/_{RT)} or with the corresponding linear relationship between log(I) and −ΔΔG/RT for a range of hybridization free energies;

wherein I is the hybridization intensity, A is an amplification factor, c the concentration of the target in solution and ΔG is hybridization free energy, T the experimental temperature and R the gas constant; and

wherein deviating from said logarithmic or linear relationship indicates the absence of said target nucleic acid of interest in said sample; and wherein not deviating from said logarithmic or linear relationship indicates the presence of said target nucleic acid of interest in said sample.

In certain embodiments, said method further comprises the step of estimating the concentration of the target nucleic acid of interest based on previously performed calibration experiments or by calculation based on an extended equation correlating the logarithm of the hybridization signal intensity with the hybridization free energy.

Preferably, in the method according to the present invention, reaching of thermodynamic equilibrium in respect of receiving detection intensity results may be taken into consideration.

Said receiving detection intensity results taking into consideration reaching of thermodynamic equilibrium may comprise receiving detection intensity results for hybridization of the target with a plurality of different probes obtained under hybridization conditions wherein thermodynamic equilibrium has been reached. In particular, in some embodiments, in step (d), only those hybridization intensity data are used resulting from a hybridization wherein thermodynamic equilibrium was reached. For instance, in some embodiments, the hybridization experiment of step (b) is performed under hybridization conditions wherein thermodynamic equilibrium has been reached.

The hybridization conditions may comprise one or a combination of hybridization time, probe length or temperature.

In some embodiments, step (d) of the method according to the invention comprises deriving that the hybridization has not reached equilibrium when a deviation from the linear relationship can be distinguished between parts of the logarithm of the hybridization intensity and the hybridization free energy, for a range of hybridization free energies. More particularly, step (d) may comprise deriving that the hybridization has not reached equilibrium when a deviation from a linear relationship with a slope 1/RT can be distinguished. A deviation from the linear relationship may be a deviation over 5%, more preferably over 10%, still more preferably over 25%, even more preferably over 33%. It is an advantage of embodiments according to the present invention that based on these results, experiments can be designed so as to be performed in a particular thermodynamic state. The latter may for example be performed by adjusting the hybridization time, the temperature, the probe length used, etc.

Thus, the analysis of the hybridization signals (also referred to herein as hybridization intensity) as function of the hybridization free energy may comprise determining whether measured hybridization signals or hybridization intensities correspond with thermodynamic equilibrium for the hybridization. It is an advantage of embodiments according to the present invention that based on these results, experiments can be designed so as to be performed in a particular thermodynamic state. The latter may for example be performed by adjusting the hybridization time, the temperature, the probe length used, etc. Receiving hybridization intensities may comprise receiving hybridization intensities for hybridization between the plurality of different probes and the target, the probes being selected so that a range of hybridization detection intensities for the hybridization between each possible target of the set of known possible targets and the probe is covered.

For a given target nucleic acid of interest out of a set of (known) target nucleic acids, a perfect match probe, and probes with up to two non-complementary elements may be provided, each to separate microarray spots for interaction with the target during the hybridization. The non-complementary elements may have a minimal effect on the hybridization free energy.

In preferred embodiments, step (c) comprises determining the hybridization free energy for the hybridization between a target in solution and a probe bound at a surface based on a nearest-neighbor model. It is an advantage of embodiments according to the present invention that hybridization free energy can be determined accurately for hybridization between a target initially in solution and a probe bound to a surface, such as for example may occur in microarrays.

Thus, the analysis of the hybridization signal intensity as function of the hybridization free energy may comprise determining a set of free energy parameters of a nearest neighbor model for the hybridization free energy based on said hybridization microarray experiment. It is an advantage of embodiments according to the present invention that good methods and systems are provided for setting up a model for determination of hybridization free energy between a target in a solution and a probe bound to a surface.

In particular embodiments, the methods are used to identify an unknown target nucleic acid of interest, more particularly when the target nucleic acid of interest can be one of a selection of target nucleic acids which differ from each other in only one or two nucleotides. This is the case for instance in the detection of polynucleotide variants in a sample, such as for instance for the identification of a viral strain.

In these embodiments, the method of the invention can be carried out by making, particularly in step (c), a first assumption for the target nucleic acid of interest, i.e. based on a first target nucleic acid of interest and, if the result of the method is that the first target nucleic acid of interest is not present, repeating the method based on, in step (c), making a second assumption of the target nucleic acid of interest, i.e. based on a second target nucleic acid of interest. As the methods of the present invention make use of a first and second plurality of probes, as defined herein, wherein each probe of set first plurality of probes is complementary to each target nucleic acid out of a said selection of target nucleic acids which differ from each other in only a limited number of nucleotides, the same first and second plurality of probes and thus the results from the hybridization experiments using the same first and second plurality of probes can be used. In these embodiments, the plurality of probes can be specifically selected so as to ensure that said plurality of probes comprises a perfectly matching (complementary) probe for each target nucleic acid of said selection of target nucleic acids which differ from each other in only a limited number of nucleotides. However, as the first and second plurality of probes already comprise probes which differ from the target nucleic acids in zero, one or two nucleotides, the perfectly matching probes for each target nucleic acid of said selection of target nucleic acids are already likely to be present in said plurality of probes from the start.

Accordingly, in particular embodiments, if in the step of determining the presence of said target nucleic acid of interest based on the relationship between said hybridization signals measured for said plurality of probes and said determined hybridization free energy, it is determined that said target nucleic acid of interest is not present in said sample solution, then said steps determining the hybridization free energy (i) for the hybridization between each probe of said first and said second plurality of probes and the target nucleic acid of interest or (ii) for the hybridization between each probe of said first and said second plurality of probes and said target nucleic acid of interest, based on a model for estimating or calculating the hybridization free energy as a sum of dinucleotide parameters and said step of (d) determining the presence of said target nucleic acid of interest based on the relationship between said hybridization signals measured for said plurality of probes and said determined hybridization free energy (steps (c) and (d) of the method as described herein) are repeated based on a another target nucleic acid of interest and said method comprises determining the presence of said other target nucleic acid of interest in said sample. Accordingly, in particular embodiments, in step (c), a first assumption is made that a first nucleic acid of said set of target nucleic acid is the target nucleic acid of interest and the hybridization free energy is determined based thereon. If, based on said first assumption, in step (d) said target nucleic acid of interest is determined not to be present in said sample solution, then said steps (c) and (d) are repeated based on a second assumption that a second nucleic acid of said set of target nucleic acids is the target nucleic acid of interest and determining the hybridization free energy based on said second assumption.

In certain embodiments, step (c) of the method according to the invention is repeated for each target nucleic acid out of a set of target nucleic acids. Thus, in certain embodiments, step (c) comprises determining the hybridization free energy for the hybridization between each probe of said first and said second plurality of probes and each target nucleic acid of the set of possible target nucleic acids. Advantageously, this allows in step (d) to derive the presence or concentration of the actual, but unknown, target nucleic acid of interest out of said set of target nucleic acids by detecting a predetermined relationship (as in equation [1], [2] or [3]) between the hybridization signal intensity and the determined hybridization free energy, wherein the actual target nucleic acid of interest present in the sample solution is identified as the target nucleic acid out of the set of target nucleic acids for which the relation of said hybridization signal intensity as a function of the estimated hybridization energy complies with or does not deviate from said predetermined relationship (as in equation [1], [2], or [3]).

In certain embodiments, the actual target nucleic acid of interest may be a minority target and the set of known possible target nucleic acids may comprise, besides the minority target nucleic acid, also a main target nucleic acid differing from the minority target in one or two non-complementary elements. Thus, in certain embodiments, the sample solution comprises a nucleic acid differing from the target nucleic acid of interest in one or two non-complementary elements, and which is present in a significantly higher concentration than said target nucleic acid of interest.

In certain embodiments, the actual target nucleic acid of interest may be an unknown nucleic acid differing from a main nucleic acid, which is present in high concentration, in one or two non-complementary elements, and the set of possible target nucleic acids may comprise the main nucleic acid and a set of nucleic acids differing from the main target in one or two non-complementary elements.

Advantageously, the method may be for use in detecting single nucleotide polymorphisms. In these embodiments, said target nucleic acid of interest is a DNA sequence comprising a single nucleotide polymorphism, i.e. differing from the natural DNA sequence in only one polynucleotide.

Hybridization may be performed in a microarray wherein each probe is provided to separate microarray spots.

In certain embodiments, the hybridization signals of the hybridization between a target nucleic acid with a probe may be induced by emission of a label associated with a hybrid formed by binding of the target and the probe, as well known to the skilled person. In particular embodiments, said label is associated with said probes.

Another object of the present invention provides a system for determining in a sample solution the presence or concentration of a target nucleic acid of interest, the system comprising:
a receiving means adapted for receiving hybridization signal data;
an analyzing means adapted for (i) determining the hybridization free energy for the hybridization between each probe of a first and a second plurality of probes and a target nucleic acid of interest of a set of target nucleic acids, based on a model for estimating or calculating the hybridization free energy as a sum of dinucleotide parameters; and

(ii) determining the presence of said target nucleic acid of interest based on the relationship between said hybridization signals and said determined hybridization free energy, particularly by comparing the relationship between said hybridization signals and said estimated or determined hybridization free energy with the logarithmic relationship

I=A. c. e^{(-ΔG}/_{RT)} or with the corresponding linear relationship between log(I) and −ΔG/RT (as in equation [3]);

with: I the hybridization intensity, A an amplification factor, c the concentration of the target in solution, ΔG the hybridization free energy, T the experimental temperature and R the gas constant;

wherein deviating from said logarithmic or linear relationship indicates the absence of said target nucleic acid of interest in the sample; and wherein not deviating from said logarithmic or linear relationship indicates the presence and/or concentration of said target nucleic acid of interest in the sample;

wherein said target nucleic acid of interest in said sample solution is a target nucleic acid of a set of target nucleic acids, wherein each of said target nucleic acids of said set of target nucleic acids differ from each other by one or two nucleotides;

wherein each probe of said first plurality of probes comprises a nucleic acid that is perfectly complementary to one nucleic acid of the set of target nucleic acids which differ from the target nucleic acid of interest in only one or two nucleotides; and

wherein each probe of said second plurality of probes comprises a nucleic acid that is non-perfectly complementary to each of said set of target nucleic acids which differ from the target nucleic acid of interest in only one or two nucleotides, the probes being selected so that a range of hybridization intensities for the hybridization between each nucleic acid of the set target nucleic acids and the probe is covered.

Particularly, the hybridization signal data is obtained from a hybridization experiment, wherein the sample solution is contacted with each probe of said first and second plurality of probes, and, subsequently a hybridization signal, in particular the intensity of the signal, is measured for each probe with the sample solution.

The present invention also relates to a method for determining hybridization free energy for the hybridization of a target initially in solution and a probe bound to a surface, the method comprising performing a hybridization microarray experiment using a plurality of microarray spots, wherein for a given target a perfect match probe, and probes with up to two non-complementary elements are provided to separate microarray spots for interaction with the target during the hybridization microarray experiment, determining a set of parameters of a nearest neighbor model for the hybridization free energy based on said hybridization microarray experiment and applying the nearest neighbor model for determination of the hybridization free energy. The present invention further relates to a method for performing hybridization, the method comprising performing hybridization between a target initially in solution and a probe bound to a surface, and applying a dehybridization step for removing cross-hybridized targets, wherein the dehybridization step is performed during a dehybridization time equal to a relaxation time of a hybridization process between a target and a probe having one or two non-complementary elements, the relaxation time being determined using a method for analyzing hybridization as described above.

The present invention also relates to a controller adapted for controlling hybridization experiments, the controller being adapted for performing hybridization between a target initially in solution and a probe bound to a surface, and for applying a dehybridization step for removing cross-hybridized targets, wherein the dehybridization step is performed over a dehybridization time equal to a relaxation time of a hybridization process between a target and a probe having one non-complementary element, the relaxation time being determined using a method for analyzing hybridization as described above.

The present invention furthermore relates to a method for analyzing hybridization, the method comprising receiving hybridization intensity data for hybridization between a target initially in solution and at least one probe bound to a surface, and analyzing the hybridization intensity as function of the hybridization free energy, the method optionally taking into consideration reaching of thermodynamic equilibrium determined using a method for analyzing hybridization as described above.

The present invention also relates to a hybridization kit for hybridization measurements for identifying an actual target out of a set of known possible targets, the hybridization kit comprising a microarray having a plurality of microarray spots each of them comprising a probe, the plurality of different probes being selected so that the corresponding hybridization covers a range of hybridization detection intensities for the hybridization of each possible target of the set of known possible targets.

The present invention furthermore relates to the use of a method for analyzing hybridization for designing a hybridization kit.

Furthermore, another object of the present invention provides a computer program product for performing, when executed on a computing device, one or more of the steps of the method according to the present invention, as described herein. It also encompasses a machine readable data storage device storing such a computer program product and transmission thereof over a local or wide area network.

Particular and preferred aspects of the invention are set out in the accompanying independent and dependent claims. Features from the dependent claims may be combined with features of the independent claims and with features of other dependent claims as appropriate and not merely as explicitly set out in the claims. These and other aspects of the invention will be apparent from and elucidated with reference to the embodiment(s) described hereinafter.

### BRIEF DESCRIPTION OF THE DRAWINGS

FIG. 1 illustrates a graph expressing the measured intensities in a hybridization experiment using a plurality of different probes bound to a surface and a target as function of the hybridization free energy whereby thermodynamic equilibrium has not been reached for all probe spots, as can be used in an embodiment according to the present invention.

FIG. 2 illustrates a graph as in FIG. 1, whereby thermodynamic equilibrium has been reached for all probe spots, as can be used in an embodiment according to the present invention.

FIG. 3A and FIG. 3B shows an example of a graph of detection intensity results as function of the hybridization free energy for detection of a target from a known set of targets, whereby FIG. 3A illustrates the results when the supposed target used for the graph is the actual target that was in the sample, whereas FIG. 3B illustrates the results when the supposed target used for the graph is not the actual target that was in the sample, as can be obtained in an embodiment according to the present invention.

FIGS. 4A-4F illustrate graphs for analysis in a method for detecting a minority target differing a single nucleotide from a main target, as can be obtained in an embodiment according to the present invention.

FIG. 5 shows a flow chart for an exemplary method for performing hybridization according to an embodiment of the present invention.

FIGS. 6A and 6B indicate correlation plots for the total intensities (a) and the median intensities (b) in replicated hybridization experiments of a first particular example illustrating features of embodiments according to the present invention.

FIGS. 7A and 7B illustrate a plot of intensities (a) as function of −ΔΔG ₛₒₗ for different concentrations and a plot of predicted behavior (b) based on the Langmuir model for a first particular example, illustrating features of embodiments according to the present invention.

FIG. 8 shows ratios of intensities and perfect match intensities as function of −ΔΔG _{μarray} for a first particular example, illustrating features of embodiments according to the present invention.

FIGS. 9A-9D show plots of I/I*ₚₘ as function of the nearest neighbor fitted −ΔΔG _{μarray} for a first particular example, illustrating features of embodiments according to the present invention.

FIG. 10 shows a comparison of data in tables 4 and 5 between ΔΔG ₛₒₗ and ΔΔG _{μarray} for a first particular example, illustrating features of embodiments according to the present invention.

FIG. 11 shows a plot of the intensity divided by concentration as function of ΔΔG for particular experiments of a second particular example with a different target concentration, illustrating features of embodiments according to the present invention.

FIG. 12 illustrates the three state model for hybridization in DNA microarrays, as can be used in embodiments according to the present invention.

FIG. 13 illustrates a numerical solution of a fraction of hybridized probes for the three state model for a second particular example of hybridization, illustrating features of embodiments according to the present invention.

FIGS. 14A-14D show a plot of the intensity as function of ΔΔG for particular experiments of a second particular example at different hybridization times, illustrating features of embodiments according to the present invention.

FIGS. 15A-15D show a plot as shown in FIGS. 14A-14D for hybridization with a shorter target sequence, illustrating features of embodiments according to the present invention.

FIG. 16 represents a flowchart showing the basic steps of the algorithm for the determination of the sequence of a target nucleic acid according to an embodiment of the present invention. tₙ is the hypothesis for the target sequence generated at the n^{th} iteration. The outputs are either a unique sequence [block (b)] or a mixed sequence composed by two sequences [block (e)] depending on the nature of the I-ΔΔG plots.

Table 1 illustrates the oligos used as target in the four different hybridization experiments of a first particular example illustrating features of embodiments according to the present invention.

Table 2 illustrates the design of the probe set used in a first particular example illustrating features of embodiments according to the present invention.

Table 3 illustrates the target conditions per microarray used in experiments of the first particular example illustrating features of embodiments according to the present invention.

Table 4 illustrates free energy differences parameters obtained from fitting microarray data to an equation expressing the logarithmic ratios of the intensities with the perfect match intensities for the first particular example, illustrating features of embodiments according to the present invention.

Table 5 illustrates data as in table 4, using the nearest neighbor parameters obtained from melting experiments in solution for the first particular example, illustrating features of embodiments according to the present invention.

Table 6 illustrates targets and probe sequences used in a second particular example, illustrating features of embodiments according to the present invention.

The drawings are only schematic and are non-limiting. In the drawings, the size of some of the elements may be exaggerated and not drawn on scale for illustrative purposes. Any reference signs in the claims shall not be construed as limiting the scope. In the different drawings, the same reference signs refer to the same or analogous elements.

### DETAILED DESCRIPTION OF THE INVENTION

Where in embodiments of the present invention the term “equilibrium” is used, reference is made to thermodynamic equilibrium describing a situation wherein a steady state is obtained such that the number of conventional target-probe bindings does not substantially change over time. The term “non-equilibrium” or “non-equilibrium effects” is used to refer to occurrence of a target-probe binding state that may change over time.

Where in embodiments of the present invention the term “free energy” is used, reference is made to the Gibbs free energy (ΔG), referring to the thermodynamic potential that measures the “useful” energy obtainable from an isothermal isobaric thermodynamic system change.

Where in embodiments analysis is performed as function of hybridization free energy (ΔG), such as when considering the hybridization intensity as a function of the (estimated) hybridization free energy (ΔG), e.g. as in equation [1], [2] or [3], this includes analysis as function of ΔΔG being the free energy difference between a perfect matching hybridization and an hybridization where the probe sequences have one or more internal mismatches.

The term hybridization used in embodiments according to the present invention refers to nucleic acid hybridization. This refers to the process of establishing a non-covalent sequence-specific interaction between two or more complementary strands of nucleic acids into a single hybrid. The strands of nucleic acids that may bind to their complement can for example be oligonucleotides, DNA, RNA or PNA. Nucleotides form the basic components of the strands of nucleic acids. Hybridization comprises binding of two perfectly complementary strands (in the Watson-Crick base-pairing senses), but also binding of non-perfect complementary strands. With a non-perfect complementary strand reference may be made to strands having a small number of non-complementary elements such as one, two or more non-complementary elements, preferably one or two non-complementary elements. In principle there is no limit to the number of non-complementary elements but the more non-complementary elements, the easier these are detectable and thus the less it is required to have dedicated methods for detecting these. In some applications the latter is un-wanted and referred to as cross-hybridization. Non-perfect complementary strands can contain different types of non-complementary elements like e.g. mismatches or loops, or any local alteration of binding properties. Non-complementary elements thereby may have a small effect on the free energy. The current invention is not limited to a particular type, but for clarity the examples given further below deal with small number of mismatches. Such a small number of mismatches may e.g. include one, two or more nucleotides that are mismatched.

Hybridization may occur between strands that both are in solution, but in the present embodiments, the hybridization envisaged is the interaction between a strand that initially is in a sample solution and a strand that is bound to a surface.

Where in embodiments of the present invention reference is made to the term “probe” a substance is envisaged that allows detection or identification of another substance in a sample. The probe may for example be a strand of nucleic acids, oligonucleotides, DNA, RNA or PNA (partially) complementary to the strand of interest in the sample solution, e.g. referred to as “actual target” or target or “actual target nucleic acid of interest” or “target nucleic acid of interest”. Object of interest that may be present in a sample solution and for which a check may be performed may be referred to as a “possible target” or “possible target nucleic acid”.

In some embodiments according to the present invention, reference is made to a minority target (or minority target nucleic acid) and a main target (or main target nucleic acid), the minority target being a target different from the main target, e.g. being slightly different, and being present in a concentration substantially smaller than the main target. In particular embodiments, the target nucleic acid may be a mutant polynucleotide, mutant or sequence variant of a (main) nucleic acid or mutant, i.e. a polynucleotide having a sequence which differs from the sequence of other nucleic acid (potentially) present in a sample (and potentially in higher concentrations) in one or more, preferably a limited set of nucleotides. It will be understood by the skilled person that the term “mutant” is not limited to sequences which are the result of a change in the target nucleic acid in a specific organism, tissue or cell but also include naturally occurring (i.e. evolutionary) sequence variants. Typically, these differences or mutations are located within a certain subsequence of the target nucleic acid. In particular embodiments, the mutant nucleic acid only differs from another target nucleic acid in one or more nucleotides within said subsequence.

The methods of the present invention make use of two different “pluralities” of probes. A “plurality of probes” as used herein refers to a set of at least 1000, more particularly at least 200, at least 500 nucleic acid probes, of the same length, the sequence of which are all different from each other. The number of probes will in part be determined by the length of the probes and the number of mismatches envisaged. This is illustrated here below. The methods of the present invention make use of a “complementary” and a “non-complementary” plurality of probes. Both of these are based on a set of different target nucleic acids which, except for the sequence differ from the target nucleic acid of interest in only one or two nucleotides. The complementary plurality of probes (also referred to herein as a “first plurality” of probes is a set of probes wherein each probe comprises a nucleic acid of which the sequence is perfectly complementary to one nucleic acid of a set of target nucleic acids which differ from the target nucleic acid of interest in only one or two nucleotides. The “non-complementary” plurality of probes (also referred to herein as the “second plurality” of probes is a set of probes wherein each probe comprises a nucleic acid that is non-perfectly complementary to each of said set of target nucleic acids which differ from the target nucleic acid of interest in only one or two nucleotides. In this way a set of probes is generated for which a range of hybridization intensities for the hybridization between the target nucleic acid of interest and the probes is covered.

The probes which make up the non-complementary plurality of probes are designed by introducing one or two mismatches in the sequence corresponding to the target nucleic acid of interest. In particular embodiments, said mismatches are not inserted at the terminal regions of the probes. More particularly, the mismatch is not inserted within 3, 4, 5 or more nucleotides from the end of the probe. In particular embodiments, if a probe of said second plurality of probes comprises two mismatching nucleotides, the non-matching nucleotides are separated by at least 5 nucleotides.

In particular embodiments, the plurality of probes is selected such that the probes hybridize to a sequence of about 15 to about 50 nucleotides of said target nucleic acids, preferably of about 15 or 35, more particularly 20 to 30 nucleotides. In particular embodiments, this implies that the length of the probes is between 15 to 50, such as between 15-35 or about 20-30 nucleotides long. However, in particular embodiments, non-hybridizing nucleotides may be added to each of the probes at the 3′ or 5′ end, such as a poly-A tail. In these embodiments, the features of the probes with respect to the complementarity to the target nucleic acid are features of the “hybridizing sequence” of the probes. It will be understood by the skilled person that while the target nucleotides and probes as described herein are envisaged to be of the same length, the target nucleotide of interest as present in the sample may be comprised within a larger polynucleotide. For instance for a probe length of 30 nucleotides, it can be envisaged to introduce mismatches from nucleotide 6 to nucleotide 25. For one mismatch, there are three different possible mismatching nucleotides and 20 available positions, hence in total 60 single mismatch sequences. A similar counting for double mismatches (which are at least 5 nucleotides apart) yields 945 different sequences.

In the methods described herein, detection of hybridization typically may be performed by a marker associated with the formed hybrid, such as for example a radio-active marker or a fluorescence marker, or other markers known in the art, embodiments of the present invention not being limited thereto. Typically in hybridization experiments, intensity of the radiation or fluorescence provided by the markers is detected and representative for the number of hybrids formed. Thus, in certain embodiments, the hybridization signal may be induced by emission of a label associated with a hybrid formed by binding of a target nucleic acid to the probes. Suitable fluorescence markers for the present methods include, but are not limited to, Cy3 and Cy5, which are dyes of the cyanine dye family, the invention not being limited thereto.

The markers or labels may be associated to the probes or to nucleic acid of interest in the sample solution prior to or after hybridization. In some embodiments, a fluorescent dye or other marker compound may be associated directly to the target nucleic acid of interest. In other embodiments, the marker compounds may be associated to the target nucleic acid in an indirect manner, for example via a “barcode”, which is a nucleotide sequence having a hybridization sequence which is complementary to a tail sequence which is present on said at least one target nucleic acid of interest, thereby allowing hybridization between the barcode and target, and therefore indirect coupling of the fluorescence marker or other marker to the target. More particularly, the strand hybridizes to a tail sequence outside the target sequence of the target nucleic acid, such that it does not significantly interfere with the hybridization between the target nucleic acid and the probes. In particular embodiments, all nucleic acids present in the sample (including the target nucleic acid of interest, if present) are amplified and tagged by a label according to methods which are well-established in the art.

However, detection of hybridization may also be performed without using markers, as is known by the skilled person. For example, probe-target hybridization may also be detected based on mass measurements, surface plasmon resonance measurements, impedance measurements, etc. Accordingly, the present methods are not limited to the detection of hybridization intensity using markers.

In the methods envisaged herein, the probes may be provided on a solid surface or in solution. In preferred embodiments, the probes are provided on a surface. Although the probes may be provided on any type of carrier, it is preferred that the probes are provided on a microarray. Thus, in particular embodiments of the methods described herein, the probes of the probe sets are provided on separate spots of a microarray. A microarray as a hybridization platform contains a large number of probes which are immobilized on a solid surface. The probes are provided in spatially separated spots, wherein each spot comprises one (and only one) type of probe. Typically, each spot comprises only a few picomoles of each probe. A spot is a local space on the microarray slide that contains a large number of identical sequences corresponding to a certain type of probe in the probe set. Therefore, each spot represents a single type of probe. Each of these identical sequences within a spot is supposed to be hybridized to a floating target sequence depending on the affinity between the two sequences. This affinity is sequence dependent and determines the fraction of hybridized probes in a spot. In preferred embodiments, the different probe sets are all provided on the same microarray. This facilitates comparing the hybridization intensities for various probe sets. Typical microarrays comprise hundreds or even thousands of spots. A plurality of microarray platforms suitable for use in the present methods is commercially available.

Examples and embodiments of the present invention may be illustrated using a particular platform for microarrays, being the Agilent platform from Palo Alto. Nevertheless, it is to be noticed that the concepts and features set forth in embodiments according to the present invention are not limited to this particular platform but can be applied mutatis mutandis to other platforms, such as for example the GeneChips platform from Affymetrix or CodeLink Bioarray platform from Amersham Biosciences. Whereas the particular fitting parameters for the model used may be platform dependent, the principles and features of the methods set out in embodiments of the present invention can be applied to these and other platforms. Furthermore, embodiments of the present invention are not limited to microarrays, but may be applied for different hybridization measurements. In particular embodiments, the probes may be provided in solution. In such embodiments, each probe may be provided in a separate solution.

The present invention relates to a method and system comprising determining hybridization between at least one target nucleic acid in a sample solution and a plurality of probes, particularly said probes bound at a surface, particularly in order to detect the presence or concentration of said target nucleic acid in the sample solution. The system and method of the present invention is especially suitable for analyzing hybridization in a microarray, particularly for detecting the presence or concentration of a target nucleic acid of interest in solution out of a set of target nucleic acids, although the invention is not limited thereto. Embodiments of the present invention may also be applied for or assist in purification applications or for selection of sequences out of a mixture, in diagnostics, in applications for detecting small mutations, in applications for detecting viruses, etc.

In the method and system according to the present invention, the probe-target affinities for the hybridization between a target nucleic acid and a plurality of non-perfectly matching probes are quantified and used to derive the presence and/or concentration of one or more target nucleic acids of interest of a set of target nucleic acids, by checking this data against the isotherm expected from equilibrium thermodynamics, in particular against equations [1] and [2] above. To this end, the plurality of probes considered in the present invention are selected to comprise a perfectly matching probe for each target nucleic acid out of said set of target nucleic acids, and a plurality of probes containing non-complementary elements, e.g. mismatches, with a target nucleic acid of interest in such a way that a sufficient range of hybridization intensities and corresponding ΔG is covered. Typically, the methods and systems will involve contacting the sample believed to comprise the target nucleic acid of interest with the plurality of probes and detecting the hybridization intensities for the different probes.

An object of the present invention provides a method for determining in a sample solution the presence of a target nucleic acid of interest, wherein the target nucleic acid of interest differs from one or more other target nucleic acids forming a set of target nucleic acids by only one or two nucleotides, the method comprising the steps of:

(a) providing
a first plurality of different probes wherein each probe comprises a nucleic acid that is perfectly complementary to one nucleic acid of a set of target nucleic acids which differ from the target nucleic acid of interest in only one or two nucleotides; and
a second plurality of different probes, wherein each probe comprises a nucleic acid that is non-perfectly complementary to each of said set of target nucleic acids which differ from the target nucleic acid of interest in only one or two nucleotides,

(b) performing a hybridization experiment, said hybridization experiment comprising contacting the sample solution with each probe of said first and second plurality of probes, and measuring a hybridization signal for each probe with the sample solution;

(c) determining the hybridization free energy for the hybridization between each probe of said first and said second plurality of probes and the target nucleic acid of interest, based on a known (mathematical) model for estimating the hybridization free energy as a sum of dinucleotide parameters; and

(d) determining the presence of said target nucleic acid of interest based on the relationship between said hybridization signals measured for said plurality of probes in step (b) and said hybridization free energy determined in step (c).

In particular embodiments it can be envisaged that the methods comprises determining which of the set of target nucleic acids is present in the sample and that step (c) comprises determining the hybridization free energy for the hybridization between each probe of said first and said second plurality of probes and each nucleic acid of the set of target nucleic acids which differ from the target nucleic acid of interest in only one or two nucleotides, based on a known (mathematical) model for estimating the hybridization free energy as a sum of dinucleotide parameters; and step (d) comprises determining the presence of said target nucleic acid of interest based on the relationship between said hybridization signals measured for said plurality of probes in step (b) and said hybridization free energy determined in step (c).

According to embodiments of the present invention, the method comprises obtaining hybridization signal data, representing the hybridization intensities for hybridization of the target nucleic acid with a plurality of probes, the probes being selected so that a range of hybridization intensities for the hybridization between the target nucleic acid and the probe is covered, or stated differently, wherein the plurality of probes have a different hybridization free energy for the at least one target nucleic acid. Hybridization may be tested or performed for each probe in a single spot, e.g. in a microarray spot. The hybridization signal intensity may be obtained based on label emission from a label associated with a hybrid formed between the probe and a target nucleic acid during hybridization.

In a particular embodiment, the invention thus also envisages the synthesis and selection of a suitable first and second plurality of probes. In particular, the hybridization sequence of the first plurality of probes is determined by the set of target nucleic acids, with the hybridization sequence of each probe being perfectly matching one target nucleic acid of said set of target nucleic acid. The hybridization sequence of the second plurality of probes comprises at least one, preferably one or two mismatching nucleotides to each target nucleic acid of said set of target nucleic acids. Advantageously, said mismatches are not inserted at the terminal regions of the hybridization sequence of a probe of said second plurality of probes, i.e. the mismatch is not inserted within 3, 4, 5 or more nucleotides from the end of the probing or hybridization sequence. Preferably, if a probe of said second plurality of probes comprises two mismatching nucleotides, the non-matching nucleotides are separated by at least 5 nucleotides.

In particular embodiments, the first and second plurality of different probes (with hybridization sequence length N, wherein N is typically ranging from about 15 to about 50 nucleotides, preferably between about 20 to about 30 nucleotides) for one target nucleic acid of interest out of said set of target nucleic acids may be generated and selected as follows:
one perfect match (PM) probe, wherein the hybridization sequence of the probe is perfectly complementary with the target nucleic acid of interest;
a set of probes in which all possible single mismatches excluding sites at 5 nucleotide distances from the end nucleotide of the hybridization sequence, to avoid terminal mismatches. This gives a total of 3(N-10) sequences, where the factor 3 counts the three possible mismatching nucleotides.
a set of probes in which all possible double-mismatch sequences are generated under the constraint that two mismatches cannot be closer than 5 nucleotides, so 9(N-16)(N-15)/2 sequences are obtained.

The range of hybridization intensities implies that a range of hybridization free energies is covered. Obtaining a set of hybridization intensity data may comprise obtaining hybridization intensities by actually measuring the hybridization intensity signals, thus including the step of performing a hybridization experiment, or the step of the detection or measurement step of the hybridization, or it may comprise receiving data via an input port for processing. In hybridization based methods, the hybridization intensity is a value representing the fraction of a certain probe which is hybridized. As used herein, the term “hybridization intensity” or “hybridization signal” refers to the intensity of hybridization with a given, individual probe as measured during the experiment.

In certain embodiments, the step of obtaining a set of hybridization intensity data may encompass performing a hybridization experiment, particularly may encompass providing a plurality of different probes applied to different spots in a microarray. Each probe thereby is bound to a surface, e.g. a microarray spot surface. Obtaining a set of hybridization intensity data furthermore may encompass providing a sample solution comprising at least one target nucleic acid of interest of a set of target nucleic acids, wherein the target nucleic acid of interest differs from one or more other target nucleic acids by only one or two nucleotides, to the surface to which the probes are linked and allowing the hybridization process to take place. Such hybridization may be platform dependent but may typically acquire at least 15 hours.

The sample solution typically comprises at least one target nucleic acid of interest out of a set of target nucleic acids, and may be prepared using standard methods known in the art. This may include extracting DNA or other polynucleotides from a sample of interest, followed by amplification of certain fragments within the extracted DNA. Typically, amplification is performed using PCR (polymerase chain reaction). However, this results in double stranded DNA, whereas single-stranded DNA is preferred for the present methods. Indeed, hybridization of double-stranded DNA with nucleic acid probes is hampered by competition between the complementary non-target strand and the probe. Such competition can be avoided by degradation of the complementary strands, for example using lambda exonuclease. Lambda exonuclease is a processive enzyme that acts in the 5′ to 3′ direction, catalyzing the removal of 5′ mononucleotides from duplex DNA. The preferred substrate is 5′-phosphorylated double stranded DNA. Accordingly, in certain embodiments, the preparation of the sample solution may comprise the steps of:
extracting DNA from a sample of interest;
amplification of one or more target nucleic acids contained in said DNA using a primer having a phosphate modification at its 5′ end; and
digesting said primer using lambda exonuclease.

Contacting the sample solution to the plurality of probes is typically performed under conditions suitable for hybridization of said target nucleic acid to said probes. Given the similarity between the possible known target nucleic acids, these hybridization conditions are suitable for hybridization with the perfect match probes of the first plurality of probes and with the probes of the second plurality of probes. The skilled person understands that the hybridization time used before measurement of the hybridization, or factors related thereto such as the length of the probes or the temperature, may play an important role in obtaining accurate results. With the formation of hybrids, typically a marker may be associated. Such markers may for example allow optical detection of the hybridization, although the invention is not limited thereto. The hybridization intensity data may encompass emission intensities of markers associated with hybrids formed during hybridization.

As indicated above, in thermodynamic equilibrium the hybridization between a target nucleic acid initially in the sample solution and a probe bound to a surface is characterized by the hybridization free energy (ΔG). This hybridization free energy ΔG expresses the free energy difference between the bound probe-target state (double stranded) and its unbound state. A method for determining such hybridization free energy (ΔG) values in the case of hybridization between a target initially in solution and a probe bound to a surface will be discussed below.

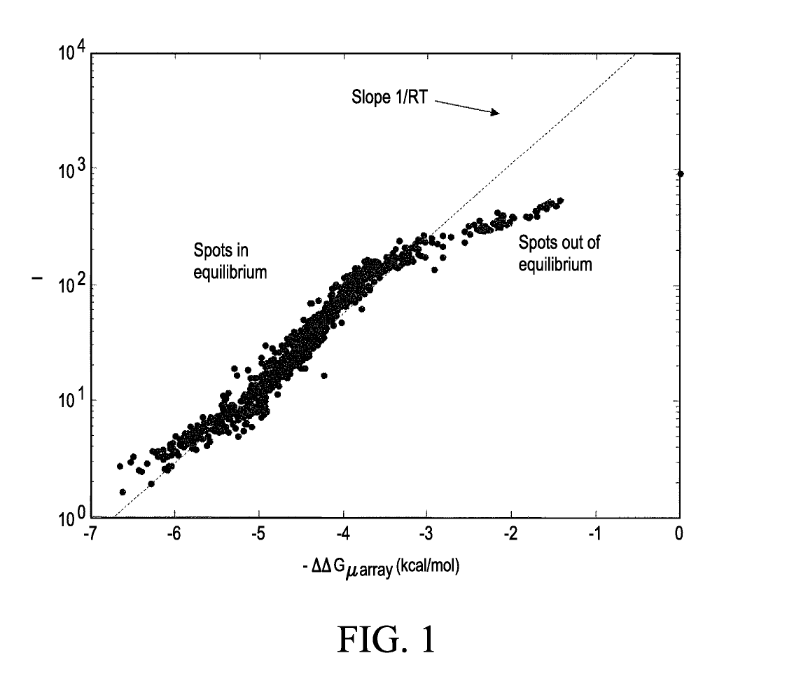

Step (d) of the method as described above generally comprises the analysis of the measured hybridization signals or intensities as function of the hybridization free energy, ΔG, particularly as determined in step (c). One exemplary way of analyzing the measured intensity as function of the hybridization free energy is discussed with reference to FIG. 1, although embodiments of the present invention are not limited thereto.

Using the plurality of different probes as described above, particularly the first and second plurality of different probes, the hybridization experiment yields an intensity value for each individual probe. Evaluating the measured hybridization intensity as function of the hybridization free energy, for example as function of the difference of hybridization free energy (ΔΔG ) with respect to the perfect match free energy for a (possible) target nucleic acid of interest, allows for providing additional information regarding not only the target nucleic acid of interest, but also on the hybridization process itself and the hybrid, target and/or probe. As will be discussed later, such analysis may take into consideration reaching a thermodynamic equilibrium, for example, the analysis may take into consideration that the hybridization is not in equilibrium yet. The analysis may comprise an analysis of the logarithm of the hybridization intensity results as function of hybridization free energy for a range of hybridization free energies.

In particular embodiments, step (d) comprises determining whether said relationship between said hybridization signals measured for said plurality of probes in step (b) and said hybridization free energy determined in step (c) corresponds to the logarithmic relationship for:

I=A. c. e^{(-ΔG}/_{RT)} (Equation [2]) or with the linear relationship between log(I) and −ΔG/RT, as represented by Equation [3];

wherein I is the hybridization intensity, A is an amplification factor, c is the concentration of the target in solution and ΔG is hybridization free energy, T is the experimental temperature and R is the gas constant; and

wherein deviating from said logarithmic or linear relationship indicates the absence of said target nucleic acid of interest in the sample; and wherein not deviating from said logarithmic or linear relationship indicates the presence and/or concentration of said target nucleic acid of interest in the sample.

The analysis may thus comprise an analysis of the logarithm of the intensity data [log(I)] as function of hybridization free energy (ΔG) for a range of hybridization free energies, such as by plotting the logarithm of the hybridization intensity values vs the hybridization free energy and assessing whether one linear relationship or a deviation therefrom can be distinguished between parts of the log(I) vs ΔG relation or plot. A deviation from a linear relationship may be a deviation of more than 5%, e.g. more than 10%, e.g. more than 25%, e.g. more than 33%. In particular, step (e) may comprise determining whether a linear relationship with slope 1/RT can be distinguished in the log(I) vs ΔG relation or plot. From this analysis different conclusions may be drawn, such as for example the presence of a target, the presence of a minority target, identification of one target out of a set of targets, identification that equilibrium has been reached or not, etc. In addition, such analysis may take into consideration reaching a thermodynamic equilibrium, for example, by not including the data points in the analysis, which correspond to a non-equilibrium hybridization.

In certain embodiments, step (d) comprises the steps

(d1) subtracting from all hybridization free energies determined in step (c) the hybridization free energy between said target nucleic acid of interest and its perfect match, thus obtaining a set of ΔΔG values;

(d2) comparing the relation of said set of hybridization intensity data as a function of the ΔΔG values with the logarithmic relationship

I=A. c. e^{(-ΔΔG}/_{RT)} (Equation [2]) or with the linear relationship between log(I) and −ΔΔG/RT according to Equation [3] for a range of hybridization free energies;

wherein deviating from said logarithmic or linear relationship indicates the absence of said target nucleic acid of interest in said sample; and wherein not deviating from said logarithmic or linear relationship indicates the presence of said target nucleic acid of interest in said sample.

FIG. 1 illustrates the logarithm of the hybridization intensities as function of the difference of hybridization free energy with respect to the perfect match free energy for a possible target. As shown above in FIG.1 some intensities correspond with spots where the hybridization is in thermodynamic equilibrium, while others are not. As shown in FIG. 1, expressing the logarithm of the intensity I in the hybridization experiments as function of the hybridization free energy (ΔG), only a partial confirmation of the linear relationship between log(I) and the hybridization free energy (ΔG) having a slope 1/RT (according to equation (3)) could be obtained. It can be seen that there is also a deviating regime with an approximate linear behavior between log(I) and the hybridization free energy (ΔG), at a different, i.e. smaller, slope. Furthermore, as illustrated in the applications, features such as equilibrium or not, presence of a target, concentration of target, identification of mutations, etc. may be derived in the analysis, as further illustrated under the different applications discussed below. As will be described later for a plurality of applications, the analysis also may comprise considering the above analysis for a plurality of targets and selecting the actual target based thereon.

According to embodiments of the present invention, performing the hybridization experiment and obtaining the hybridization signal data (step (b)) and/or the subsequent analysis of step (d) may take into consideration reaching of thermodynamic equilibrium, such as e.g. the fact that no thermodynamic equilibrium state has been reached already. In some embodiments according to the present invention, the step (b) may take into consideration a thermodynamic non-equilibrium state of the hybridization. The latter may for example encompass adapting the hybridization conditions such that thermodynamic equilibrium or thermodynamic non-equilibrium can be obtained. Such hybridization conditions may comprise for example the temperature at which hybridization is performed, the length of the probes used, the hybridization time used, etc. In some embodiments according to the present invention, the step (d) takes into consideration a thermodynamic non-equilibrium state for the hybridization. The latter may be performed by discarding a certain number of intensity results obtained, fitting the predetermined correlations as envisaged herein (see equations [1], [2] or [3]) to part of the data obtained. In some embodiments, it is performed by fitting different predetermined correlations to the detected intensity, one being representative for the equilibrium state and one being representative for the non-equilibrium state.

Without wanting to be bound by theory, the results obtained with methods and/or system embodiments according to the present invention could be explained by the occurrence of non-equilibrium effects. More particularly, it was surprisingly found that, although typically the assumption of thermodynamic equilibrium was made in hybridization experiments, thermodynamic non-equilibrium effects play a significant role. Experiments show that upon increase of the hybridization time, substantially over times that are conventionally used in hybridization experiments, more probes reach equilibrium. The experimental results for non-equilibrium effects could be explained by determination of the hybridization free energy (ΔG) which could be determined using a nearest neighbor model for the hybridization. The nearest neighbor model for hybridization between a target and a probe bound to a surface could be applied and model parameters could be determined due to a particular experimental design and dedicated custom microarrays, an example thereof being described further below. By extending the hybridization theory, from the two-state model mentioned above to a three state model, the form of the experimentally determined non-equilibrium effects could be explained. As shown in FIG. 1, expressing the logarithm of the measured intensity I in the hybridization experiments as function of the hybridization free energy (ΔG), only a partial confirmation of the linear relationship between log(I) and the hybridization free energy (ΔG) having a slope 1/RT could be obtained. It can be seen that there is also a deviating regime with an approximate linear behavior between log(I) and the hybridization free energy (ΔG), at a different, i.e. smaller, slope.

It is noted that values for ΔG or ΔΔG can be determined in various ways, e.g. the values may be determined experimentally or calculated. In particular embodiments, a good estimate of these values may be determined (in step (c) of the method) via known models for estimating the hybridization free energy as a sum of dinucleotide parameters, e.g. the nearest-neighbor model as known by the skilled person (see Hadiwikarta WW et al., Nucleic Acids Res. 2012, 40, e138; and references cited therein). Additionally or alternatively, the values for ΔG or ΔΔG can be determined experimentally. Thus, whereas embodiments of the invention mainly focus on systems and methods for analyzing hybridization or setting up hybridization experiments, in one aspect embodiments of the present invention also relate to a method and system for determining hybridization free energy for the hybridization of a target initially in solution and a probe bound to a surface or for setting up a nearest neighbor model for the hybridization free energy between a target initially in sample solution and a probe bound to a surface. The method thereby comprises performing a hybridization microarray experiment using a plurality of microarray spots, wherein for a given target a perfect match probe and probes with up to two non-complementary elements such as two nucleotide mismatches are provided, e.g. by printing, on separate microarray spots for interaction with the target during the hybridization microarray experiment. The method also comprises determining a set of parameters for a nearest neighbor model for the hybridization free energy based on the hybridization microarray experiment. Optionally the method may be directly applied for determination of the hybridization free energy.

Turning back to the non-equilibrium conditions for hybridization, it is to be noticed that for the applications, and their interpretation, it is advantageous to know in which regime one is measuring, since the regime influences the effect on intensity due to an additional non-complementary element, e.g. mismatch, in the target-probe duplex. If for example, one wants a probe that is as specific as possible to the perfect match target, in order to avoid that non-perfect matching targets hybridize the probe i.e. in order to avoid cross-hybridization, it is important that the measurements are performed in the regime showing a relation between the logarithmic detection intensity and the hybridization free energy with the highest slope, as this is the regime with the highest specificity.

In certain embodiments, the present invention provides a method for performing a hybridization experiment especially suitable for identifying an actual target nucleic acid of interest out of a set of known possible target nucleic acids. The method comprises obtaining hybridization intensity data for hybridization of the actual but unknown target nucleic acid of interest with a plurality of different probes, the probes comprising a first and a second plurality of probes as defined herein, and being selected so that a range of hybridization detection intensities for the hybridization between each possible target nucleic acid of the set of known possible target nucleic acids and the probe is covered. The latter directly implies that a range of free energies is covered. The method also comprises evaluating the obtained hybridization intensity values as function of the hybridization free energy for each of the possible targets of the set of known possible targets and deriving the presence or concentration of the actual target nucleic acid by detecting, for one of the target nucleic acids of said set of target nucleic acids, a predetermined relationship between the hybridization intensity and the hybridization free energy. Said predetermined relationship corresponds to the relationship expressed in equations [1], [2] or [3]. It is an advantage if reaching of equilibrium is taken into account, as described herein, although the embodiment is not limited thereto.

In particular embodiments, such as wherein the identification of the actual target of interest is desired (i.e. the actual sequence of the target of interest present in the sample solution is not known), in step (c), a first assumption may be made that a first nucleic acid of said set of target nucleic acid is the target nucleic acid of interest and determining the hybridization free energy thereon. If, based on said first assumption, in step (d) said target nucleic acid of interest is determined not to be present in said sample solution, then said steps (c) and (d) are repeated based on a second assumption that a second nucleic acid of said set of target nucleic acids is the target nucleic acid of interest and determining the hybridization free energy based on said second assumption.

In a further embodiment, the actual target nucleic acid of interest is a known minority target nucleic acid which is present together with a known main target nucleic acid which differs slightly from the minority target e.g. in one or two non-complementary elements such as by one or two nucleotide mismatches. The sample solution comprises one target nucleic acid differing from the target nucleic acid of interest in one or two non-complementary elements, which is present in significantly higher concentration than said target nucleic acid of interest. The methods as described herein allow detection of the presence of the minority target and a quantification of the relative concentration.

In another embodiment, the actual target nucleic acid of interest is an unknown target nucleic acid differing slightly from a main target nucleic acid, e.g. in one or two non-complementary elements such as by one or two nucleotide mismatches, and the set of known possible target nucleic acids comprises the main target and a set of targets differing from the main target in one or two nucleotides. The methods as described herein allow identification of the actual target nucleic acid of interest. Further details and advantages are described in the following exemplary applications and embodiments.

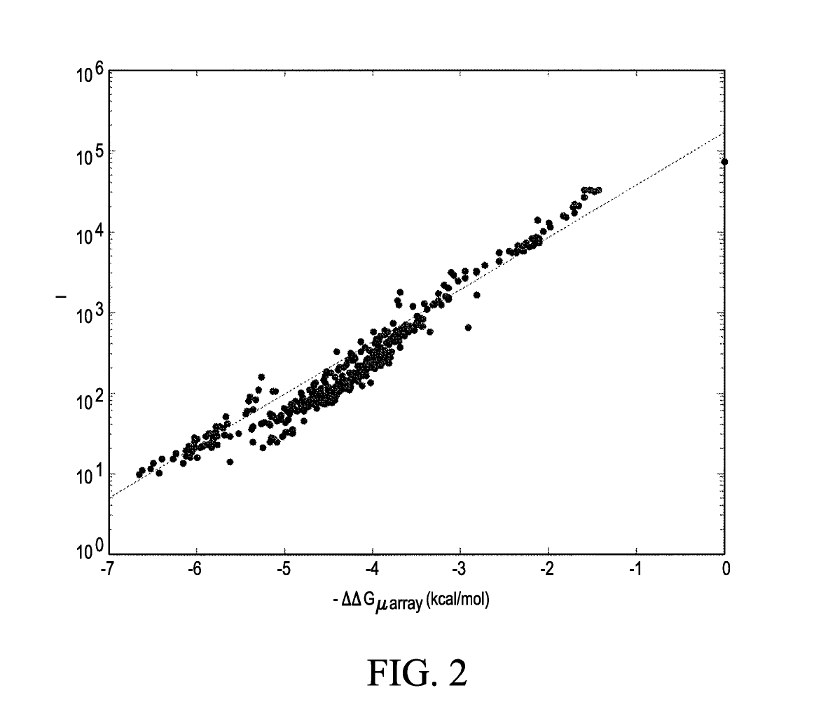

As a first exemplary application, in certain embodiments, the present invention provides a system or method for determining whether a hybridization experiment is in equilibrium or not. The method comprises performing a hybridization experiment using a plurality of different probes, whereby the different probes are selected to comprise a perfect matching probe for the target and probes containing non-complementary elements, e.g. mismatches, with the target in such a way that a sufficient range of intensities and corresponding therewith ΔG is covered. The hybridization experiment then results in an intensity value per probe. Evaluating the measured intensity as function of the hybridization free energy, for example as function of the difference of hybridization free energy with respect to the perfect match free energy for a possible target, allows for determining whether a spot has reached equilibrium or not. Spots that fulfil a predetermined relation between the logarithmic intensity measured and the hybridization free energy, e.g. that fall on a line with slope 1/RT, are in equilibrium, spots deviating therefrom are not in equilibrium. By way of illustration, as shown above in FIG. 1 some spots may be in thermodynamic equilibrium, while others are not. The intensities corresponding to spots not in equilibrium do not fulfil a predetermined relationship between the logarithmic intensity and hybridization free energy. In FIG. 1, only the spots on the bottom line in the graph correspond with spots that are in equilibrium. FIG. 2 illustrates an experiment whereby thermodynamic equilibrium is reached for all spots. The example shown in FIG. 2 is based on an example wherein the targets are shorter than in FIG. 1 (25 nucleotides instead of 30 nucleotides), allowing for quicker dynamics and consequently to an earlier reached equilibrium state.

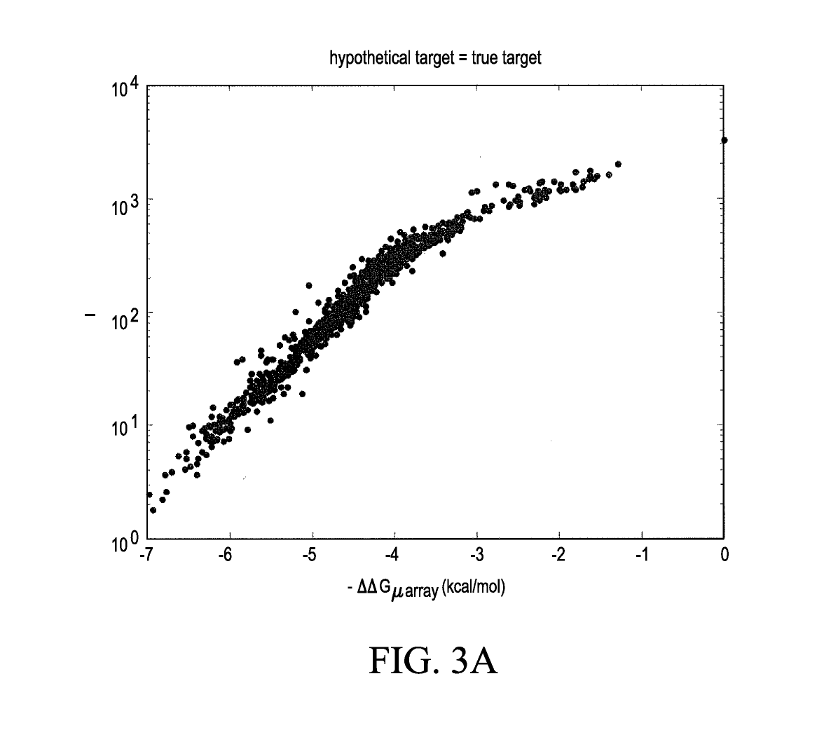

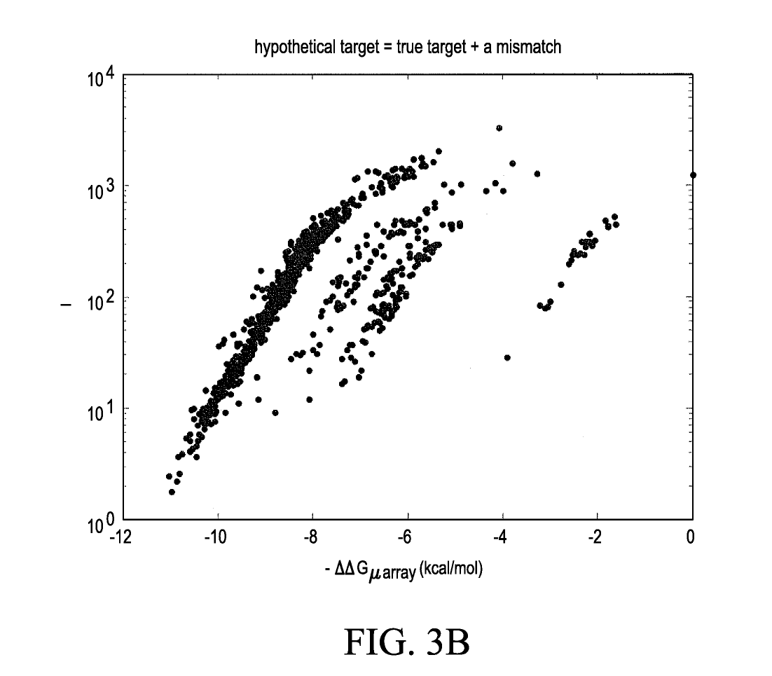

As another exemplary application, in certain embodiments, the present invention provides a system and/or method for identifying a target nucleic acid of interest out of a set of (known) target nucleic acids in a sample solution, i.e. one wants to know which of a known set of targets is in the solution. Whereas this problem could be solved by sequencing, the problem also could be solved by a hybridization experiment in line with the methods as envisaged herein. The system and/or method comprises performing a hybridization experiment using a plurality of different probes, in particular a first and a second plurality of different probes as specified herein, whereby the probes are selected to comprise, for each target nucleic acid of the set of known target nucleic acids, a perfect matching probe for the target and probes containing non-complementary elements with the target in such a way that a sufficient range of detection intensity and consequently of ΔG is covered. The hybridization may be performed in a micro-array experiment, wherein each probe occupies a single spot in the microarray and is contacted with the sample solution, although embodiments of the present invention are not limited thereto. The hybridization experiment then results in an intensity value per probe. For each possible target of the known set of targets, the obtained hybridization intensity for the plurality of different probes, in particular a first and a second plurality of probes as specified herein, is evaluated (e.g. by plotting) as function of the hybridization free energy ΔG of the possible target nucleic acid, for instance as function of ΔΔG, i.e. the difference between the obtained hybridization free energy and the hybridization free energy of the perfect match for the possible target. Based on this evaluation, identification of the actual target in the solution can be performed, as only in the case of the actual target, a predetermined correlation, as represented by equation ([1], [2] and/or [3], is detectable in the evaluation. By way of illustration, an example of such an evaluation is shown in FIG. 3A and FIG. 3B. The obtained hybridization intensity is set out as function of the difference of hybridization free energy with respect to the perfect match free energy for a possible target. The x-axis of the figure is dependent on the possible target selected (i.e. based on the assumption that a particular target nucleic of said set of target nucleic acid is present in the sample solution, which assumption is to be evaluated), as the property on the axis is function of the free energy between the probe and the possible target, i.e. function of ΔG(probe_i, possible target). FIG. 3A illustrates the obtained result for a possible (hypothetical) target nucleic acid corresponding with the actual target nucleic acid in the solution, wherein a plot of measured hybridization intensities as a function of ΔΔG results in a single curve, or stated differently, collapsing of the data points into a single curve, whereas FIG. 3B illustrates the obtained result for a possible, hypothetical target nucleic acid not corresponding with the actual target nucleic acid in the sample solution. In the latter case, no single curve is obtained. The actual target nucleic acid of interest can thus be identified as the possible target nucleic acid for which the evaluation results in a predetermined relationship between intensity measured during the hybridization experiments and the hybridization free energy for the possible target ΔG(probe_i, possible target).

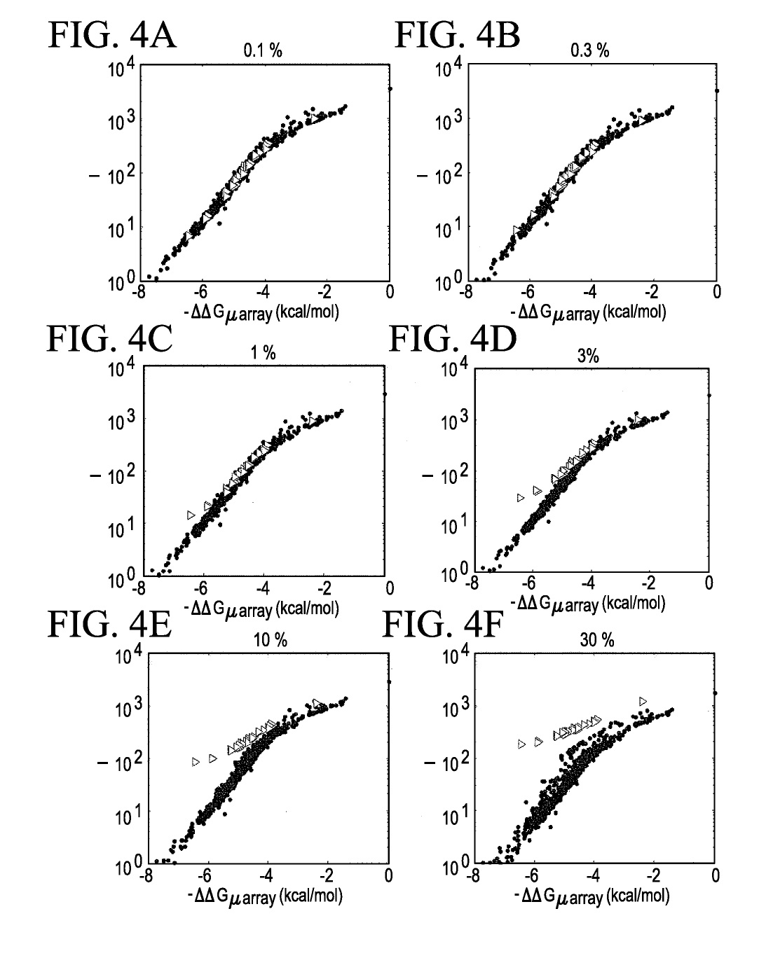

In other embodiments, the present invention relates to a system and/or method for detecting presence of and/or quantifying a minority target nucleic acid in a solution comprising a main (known) target nucleic acid being very similar to the minority target. With very similar there may be meant that there are only one or two non-complementary elements, such as one or two nucleotide difference. The method and system according to embodiments of the present invention allows deriving the presence of a minority target and/or deriving an estimate of the proportion in which the minority target is present. It is an advantage of embodiments according to the present invention that the system and method for detection of minority targets in a sample solution that differ only slightly from a main target present in the solution is sensitive. It is an advantage of embodiments according to the present invention that a detection limit for minority targets can be obtained that is substantially better than 20%, as is for example the obtainable detection limit of Sanger sequencing techniques. It is an advantage of embodiments according to the present invention that detection may be performed based on hybridization experiments. This type of analysis can be of relevance for the early diagnostics of mutations in specific genomic regions, for instance in HIV or cancer. Indeed, the analysis of a biopsy for the detection of cancer comprises both normal, wild type cells, and possible mutant cancer cells. The system and/or method comprises performing a hybridization experiment using a plurality of different probes, in particular a first and a second plurality of probes as defined above, whereby the probes are selected to comprise, for both the main target and the minority target, a perfect matching probe for the target and probes containing non-complementary elements with the target in such a way that a sufficient range of detection intensities and consequently of hybridization free energy ΔG is covered. The hybridization may be performed in a microarray experiment, although the invention is not limited thereto. The hybridization experiment results in an intensity value for each probe, thus obtaining hybridization intensity data. For the main target, the measured intensity for the plurality of different probes is then evaluated (e.g. plotted) as function of the hybridization free energy of the possible target, e.g. as function of the difference between hybridization free energy with respect to the perfect match free energy for the possible target. Based on this evaluation, the presence or absence of the minority target can be derived from whether or not a predetermined relation, corresponding to equation [1], [2] or [3]: if a single, predetermined correlation is derived, i.e. if the data collapse in a single curve, the minority target is not present within the detection limit. If there is a deviation from a single curve or predetermined correlation (i.e. separate branches appear), the minority target is present, and the proportion can be estimated. The latter may for example be performed, based on previously performed calibration measurements, or by theoretical calculation based on an extended equation correlating the logarithm of the measured intensity with the free energy ΔG. By way of illustration, a particular test experiment is shown in FIGS. 4A-4F, showing the possibilities for detecting a minority target that differs in a single nucleotide from a main target using a method as described above. Experiments were performed with samples containing a known concentration of minority target, varying over the different plots from 0.1% to 30%. In FIGS. 4A-4F, the spots that are most sensitive to the presence of the minority target are indicated as triangles. It can be seen that from 1% a 3% onwards, in the present example, detection of the minority target is possible. Such a detection limit is substantially better than what is conventionally obtained using Sanger sequencing. The experiments can also be further optimized by taking into account the non-equilibrium effect occurring in hybridization experiments, as described herein. The latter typically results in that only the lowest spots indicated by triangles are in equilibrium and therefore are most sensitive. Quantification therefore preferably is based on these lower spots.

As another exemplary application, other embodiments relate to methods and systems for the detection of a minority target nucleic acid in a sample, similar as described in the previous application, but wherein the sequence of the (minority) target is not known as such, but only differs from the main target by one or two non-complementary elements, for instance, in the present example being one or two nucleotides. The sequence of the minority target then can be derived by performing an experiment as set out above, whereby it is not a priori known which probes correspond with the minority target nucleic acid. Identification of the probes corresponding with those intensity values deviating from the predetermined correlation for intensity as function of the free energy may allow for identification of the sequence of the minority target. It is an advantage of embodiments according to the present invention that such techniques can advantageously be applied for searching single nucleotide polymorphisms. It is an advantage of embodiments according to the present invention that alternative and complementary techniques with respect to purely statistical methods are provided for tackling searching of single nucleotide polymorphisms.

The different exemplary applications described above can be combined resulting in advantageous characterization and detection methods.

Whereas the different embodiments have mainly been described as methods, also systems are encompassed.

Systems according to the present aspects typically comprise a receiving means, e.g. an input port but alternatively also a hybridization measurement setup, for receiving the detection intensity data as described above and a processing means or processor for evaluation and calculation purposes of the hybridization intensity data as described above. The system furthermore may comprise additional components for performing additional functionalities as described in the method embodiments described in the present application.

In another aspect, the present invention also relates to a method for performing hybridization, wherein first a hybridization step is performed between a target nucleic acid initially in the sample solution and a probe bound to a surface and thereafter a dehybridization step is performed for removing cross-hybridized targets. The dehybridization step is performed over a dehybridization time substantially equal to the relaxation time of a hybridization process between a target and a surface-bound probe having the unwanted non-complementary element, e.g. mismatch. The latter allows removal of such hybrids having the non-complementary element, while keeping the hybrids with perfect match. The relaxation time may advantageously be determined using a method as described above, applied to a target and a probe having the non-complementary element. An example of such a method for performing hybridization is given below by way of an exemplary application. The present aspect also relates to a controller for controlling a hybridization setup used for hybridization experiments and thus for controlling hybridization experiments as described above. The controller thus may be adapted for performing a hybridization step and for performing a dehybridization step for removing cross-hybridized targets, whereby the dehybridization time advantageously is determined using a method for analyzing hybridization as described above. The controller may comprise a processor for performing an algorithm implementing the steps of a method for performing hybridization as described above.

Thus, embodiments of the present application relate to a system or method for increasing specificity of hybridization experiments. The specificity is increased by minimizing cross-hybridization. The latter is obtained by, after a hybridization experiment has reached equilibrium, applying a dehybridization step for removing cross-hybridized targets. The dehybridization step thereby is characterized by a dehybridization time set equal to the relaxation time of the hybridization process for a target-probe complex having a non-complementary element, being a mismatch in the present example, as can be determined using a method according to an embodiment of the present invention. It may be set equal to the relaxation time of the target-probe complex having a non-complementary element having the smallest effect on the hybridization energy (with reference to a perfect match) of all unwanted non-complementary elements. The relaxation time of target-probe complexes having at least one non-complementary element differs from, typically is shorter than, the de-hybridization time of a target-probe having no non-complementary element, as the hybridization of the target-probe complex containing a non-complementary element has a lower free energy. To determine the optimal dehybridization time analysis is made of the graphs of log I as function of ΔG and selection of the dehybridization time is performed for conditions wherein the ratio of the intensity corresponding with probes having perfect match to intensity corresponding with probes having the non-complementary element is maximal. The de-hybridization may for example be performed by washing out the sample so that the target molecules are removed from the solution. The washing may be done with pure water or with water solution containing appropriated solvents. By applying the de-hybridization step, most of the cross-hybridized targets will be removed, while most of the perfect matching targets will remain on the probe-spot. Such a system or method may be applied for microarray applications, although the invention is not limited thereto. The technique also can be applied for purification, for selection of sequences out of a mixture for example as a preliminary step for sequencing experiments, etc.

In another embodiment, the present invention relates to a method for analyzing hybridization comprising receiving hybridization intensity data for hybridization between a target initially in solution and at least one probe bound to a surface, and analyzing the detection intensity results as function of the hybridization free energy. The method thereby takes into consideration the reaching of thermodynamic equilibrium which advantageously may be determined using a method for analyzing hybridization as described above.

In still another aspect of the present invention, a hybridization kit for hybridization measurements for identifying an actual target out of a set of known possible targets is described. The hybridization kit comprises a microarray having a plurality of microarray spots each of them comprising a probe out of a first and second plurality of probes, selected as described above, so that the plurality of different probes covers a range of detection intensities and corresponding therewith a range of hybridization free energies for the hybridization between each possible target of the set of known possible targets and the probes. The hybridization kit may be especially suitable for performing applications as described in the present invention, although the invention is not limited thereto. The different probes may differ from each other or from one sub-set of the plurality of different probes only slightly, being by one or two non-complementary elements such as mismatches having a small influence on the hybridization free energy.

In yet another aspect, the methods based on analyzing hybridization may be used for designing a hybridization kit, e.g. a kit as described above, although not limited thereto. The methods may in one example be used for determining whether for a given length of the probes equilibrium would be obtained and therefore may be used in the design of a hybridization kit for determining a length of the probes used. In another example, the methods may be used for determining whether a sufficient range of detection intensity and corresponding therewith a sufficient range of hybridization free energy is covered by a predetermined set of probes, and if required, the set of probes may be adjusted in view of this. Based on the above methods for analyzing hybridization, a number of design guidelines may be derived. The methods based on analyzing hybridization as described above thus also may be used as a method for calibrating or setting up a hybridization experiment, used as method for calibrating a nearest neighbor model as will be described below, as well as to analyze the hybridization experiments performed.

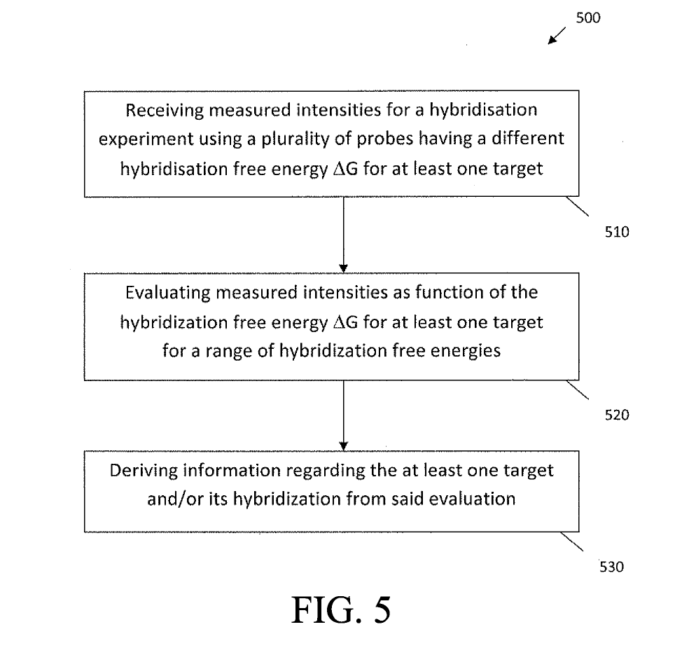

Other objects and embodiments of the present invention also relate to computer-implemented methods for performing at least part of the methods of the present invention as described herein. Embodiments of the present invention also relate to corresponding computer program products. The methods may be implemented in a computing system. They may be implemented as software, as hardware or as a combination thereof. Such methods may be adapted for being performed on computer in an automated and/or automatic way. In case of implementation or partly implementation as software, such software may be adapted to run on suitable computer or computer platform, based on one or more processors. The software may be adapted for use with any suitable operating system such as for example a Windows operating system or Linux operating system. The computing means may comprise a processing means or processor for processing data. According to some embodiments, the processing means or processor may be adapted for performing analysis of one or more hybridization experiments according to any of the methods as described above. The processor therefore may be adapted for evaluating measured hybridization intensities as function of hybridization free energies and for determining therefrom an analysis result. Performing such analysis or evaluation may comprise for example determining whether hybridization with certain probes was measured in equilibrium, determining the presence of a known target, determining the presence and/or quantity of a minority target, identifying a minority target being present, etc. Besides a processor, the computing system furthermore may comprise a memory system including for example ROM or RAM, an output system such as for example a CD-rom or DVD drive or means for outputting information over a network. Conventional computer components such as for example a keyboard, display, pointing device, input and output ports, etc also may be included. Data transport may be provided based on data busses. The memory of the computing system may comprise a set of instructions, which, when implemented on the computing system, result in implementation of part or all of the standard steps of the methods as set out above and optionally of the optional steps as set out above. By way of illustration, the present invention not being limited thereto, an exemplary flow scheme of a computer implemented method is shown in FIG. 5. The computer implemented method 500 for analyzing hybridization may comprise:

(510) receiving measured hybridization intensities for a hybridization experiment wherein the sample solution is contacted with a plurality, particularly a first and a second plurality of different probes, as defined above, having a different hybridization free energy ΔG for at least one target nucleic acid. Such receiving may be receiving data obtained from a previously/externally performed hybridization experiment via an input means. Alternatively, such receiving also may comprise obtaining the data directly through measurement during the hybridization experiment. The corresponding computing system therefore may comprise an input means for receiving input. The results also may be stored intermediately.

(520) evaluating measured intensities as function of the hybridization free energy for at least one target for a range of hybridization intensities and correspondingly a range of hybridization free energies. The range of hybridization intensities or hybridization free energies may be selected as function of the target nucleic acid to be characterized. Selection of such a range may for example be based on previously obtained results, or through trial and error. The hybridization free energy may be estimated via known models for estimating the hybridization free energy as a sum of dinucleotide parameters, particularly the nearest-neighbor model as known by the skilled person. Evaluating may comprise plotting or determining a correlation between the measured hybridization intensities and the hybridization free energy or a property based thereon or being function thereof. Depending on the application, such an evaluation may be performed for one (known or unknown) target, for a plurality of known targets that may be involved, etc. Such evaluation may be performed by an evaluation component of the processor.

(530) deriving information regarding the at least one target and/or its hybridization, based on the evaluation of step (520), e.g. on an established correlation between the hybridization intensities and the hybridization free energy. The latter may for example comprise determining the actual target nucleic acid present in the sample studied by hybridization, determining the presence and/or concentration of minority target nucleic acids present in the sample studied by hybridization, detecting single nucleotide polymorphisms of targets in the sample studied, characterising the actual target nucleic acid present in the sample studied by hybridization, determining whether the performed hybridization experiment was in equilibrium for given probes, etc. Such information may be derived based on fitting of predetermined curves, i.e. curves corresponding to equation [1], [2] or [3] to the obtained correlation, evaluating deviation from predetermined curves, determining the slope of parts of curves, etc. The obtained results may be outputted through an output means such as for example a plotter, printer, display or as output data in electronic format.

The computing system and/or a corresponding computer implemented method may also be adapted for controlling the performance of the hybridization experiments themselves, although the invention is not limited thereby.

Further aspect of embodiments of the present invention encompass computer program products embodied in a carrier medium carrying machine readable code for execution on a computing device, the computer program products as such as well as the data carrier such as dvd or cd-rom or memory device. Aspects of embodiments furthermore encompass the transmitting of a computer program product over a network, such as for example a local network or a wide area network, as well as the transmission signals corresponding therewith.

While the invention has been illustrated and described in detail in the drawings and foregoing description, such illustration and description are to be considered illustrative or exemplary and not restrictive. The invention is not limited to the disclosed embodiments.

### EXAMPLES

### Example 1

### Detection and Identification of HIV Variants

HIV samples. HIV-1 virus stocks were selected from the Janssen Diagnostics repository database, based on their known mutation profile in the region of codon 179 to codon 186 of the Reverse Transcriptase (RT) gene. This region was selected to cover key resistance mutations at position 179, 181 and 184.

Experimental protocol. The viral RNA extraction of virus stocks was carried out on an EasyMAG (bioMe'rieux, Boxtel, The Netherlands) according to the guidelines of the manufacturer. A One-Step RT-PCR amplification (One-Step Superscript III HiFi, Invitrogen, Calif., USA) was used to generate a 2.3-kb HIV-1 fragment (containing the gag-protease-reverse-transcriptase (GPRT) region) using the 3-RT (5′-CATTGCTCTCCAATTACTGTGATATTTCTCATG-3′) and 5-OUT (5′-GCCCCTAGGAAAAAGGGCTGTTGG-3′) primers. The 2.3-kb HIV-1 outer fragment generated with the GPRT one-step PCR was used as template for the asymmetric amplification of the sequence around RT codon 184. Therefore, the HIV-1_Fw_184Cy3 (5′-/Cy3/TAGAAAACAAAATCCAGAAATA-3′) and HIV-1_Rev_184 (5-TGCCCTATTTCTAAGTCAGATCC-3′) primers were used, with the fluorescent labelled forward primer HIV-1_Fw_184Cy3 in excess to generate fluorescent labelled single-stranded DNA (ssDNA) fragments of 78 bp (containing RT codon 184). Forward primer HIV-1_Fw_184Cy3 and reversed primer HIV-1_Rev_184 were used at a concentration of 1 mM and 0.1 mM, respectively, with DNA input of 2 ml in a final volume of 100 ml. The microarray experiments were performed in an Agilent platform. We considered hybridizing sequences of 25 nt, as they have been found to attain thermodynamic equilibrium after about 3 h of hybridization (in the experiments, the hybridization time is of 17 h to ensure that equilibrium was reached).

The raw microarray data were subjected to a primary quality control using the Agilent Feature Extraction Software (Version 10.7).

Microarray design. When considering the sequences with high clinical frequency obtained with Sanger sequencing from a database provided by Janssen Diagnostics, of about 350 000 patients, on a region of 25 nt of the HIV-RT centered around codon 184, it was found that the sequence with the highest frequency occurs in only 25% of the patients. The other 75% of the cases contain mutations with respect to it. This makes the HIV an excellent test model to check the validity of the method presented herein. Part of the sequences were unique sequences. Other samples are mixed, i.e. HIV viruses with sequence differences within the 25-nt window considered coexist in a single patient. Further, the concept of unique and mixed samples has to be interpreted within the limited sensitivity of Sanger sequencing. The Sanger method can detect a mixture only if the relative abundance of the low abundance sequence is >20%. For instance, a mixed sequence sample with only 10% of low abundance sequence would be detected as unique sequence sample by the Sanger method. To define a manageable and relevant diagnostic problem, each unique sample from the database was selected and ranked in decreasing order of clinical frequency. The goal was to diagnose the top 100 unique sequences and mixtures of two sequences thereof. This corresponds to the coverage of 82% of the whole database.

The 15 k custom Agilent array used in the experiment contains about 15 000 spots. The design of the probe sequences on the array was started from the 100 probes that are perfectly matching to the 100 unique sequences considered in the test (referred as the perfect match (PM) set in the design). The data analysis of the method presented herein is not solely based on the signal of perfectly matching spot, but also on cross-hybridizing signals. Indeed, signals measured from spots with one or two mismatches with respect to the target sequence are still above the lower limit of detection. Therefore, probes with one or two mismatches with respect to those in the PM set were included in the microarray, under the constraints that the mismatched nucleotide cannot be situated near the end nucleotide of the hybridization sequence, to avoid terminal mismatches, and that two mismatches cannot be closer than 5 nucleotides from each other.

The final design contains 2139 different probes replicated seven times to fill all the available spots of the 15 k microarray.

Thermodynamic assessment. The approach envisaged herein is based on the analysis of the intensities of the brightest spots and also of many ‘dimmer’ spots which are expected to carry one or two mismatches with respect to the target (mismatching sequences). If a unique target nucleic acid is present in solution, the fluorescence intensity I from different spots will be correlated according to equilibrium thermodynamics as presented in equation [3] above (for sufficiently low concentrations; wherein A is a proportionality factor, c the target concentration in solution, ΔG the hybridization free energy as a sequence-dependent measure of the affinity between probe-target sequences, R the gas constant, and T the temperature. For convenience in the rest of this example, hybridization free energies are shifted by the corresponding perfect match value. This amounts to use ΔΔG=ΔG −ΔG(PM). As described above, the same functional relationship as Equation [3], holds also for ΔΔG.

Given the possible sequences of the target nucleic acids in solution and the surface-bound probes, the ΔΔG can be obtained from the nearest-neighbor model. In practice, the target sequences are coming from a biological sample, and they are usually not known. One can, however, make a starting hypothesis about the sequence and compute the corresponding ΔΔG. If the starting hypothesis agrees with the actual sequence in the sample, the measured intensities should be distributed according to Equation [3]. Deviations from this law may have two causes: (i) The starting hypothesis is wrong; hence, the sequence in the sample is different from what originally assumed or (ii) the sample is a mixture, i.e. it contains the sequence of the original hypothesis together with other sequences. This concept is illustrated in FIG. 2 that shows plots of I versus ΔΔG in log-linear scale for which Equation (3) becomes a straight line with slope 1=RT (shown as dashed line). The two plots of FIGS. 3A and 3B are based on the same experimental data. In one case (FIG. 3A), the hypothesis matches the actual sequence in the sample. The data accurately follow Equation [3] over four orders of magnitude in the intensity scale. In the other case (FIG. 3B), the wrong hypothesis leads to an incorrect computation of the ΔΔG and deviations from the expected thermodynamic behavior. Here, the data are distributed into four distinct branches.

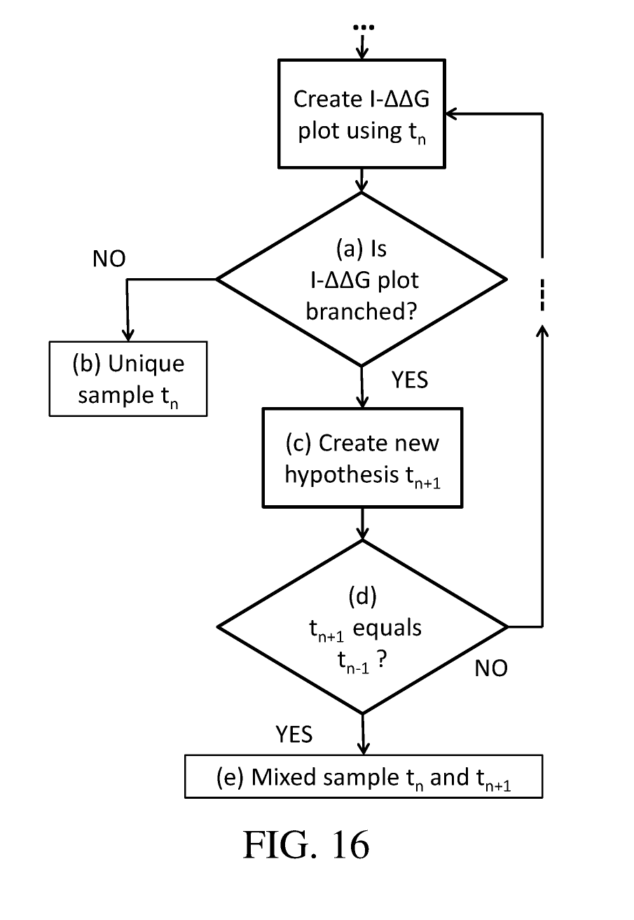

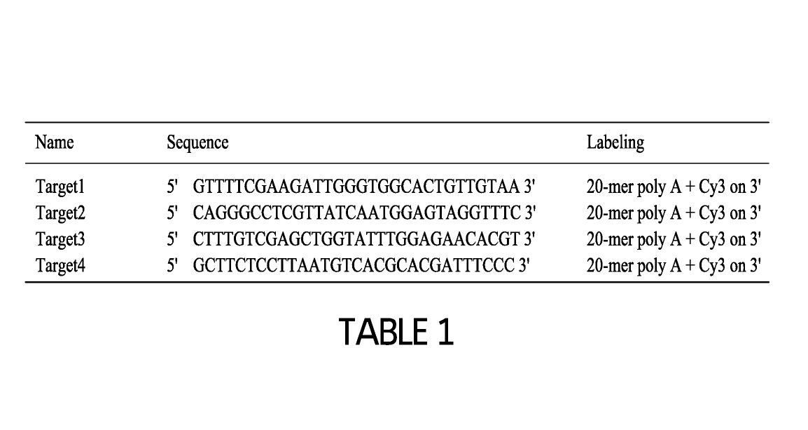

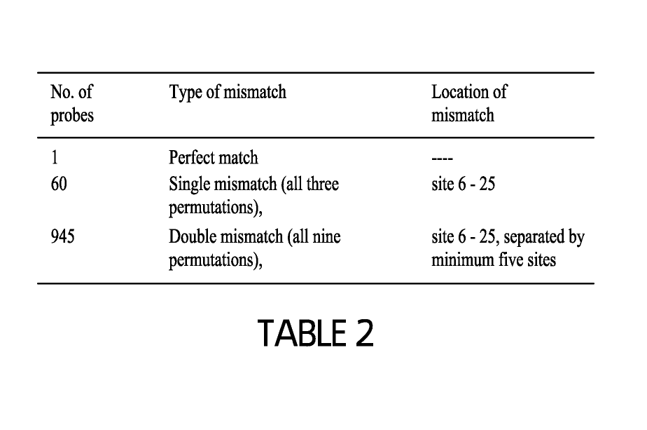

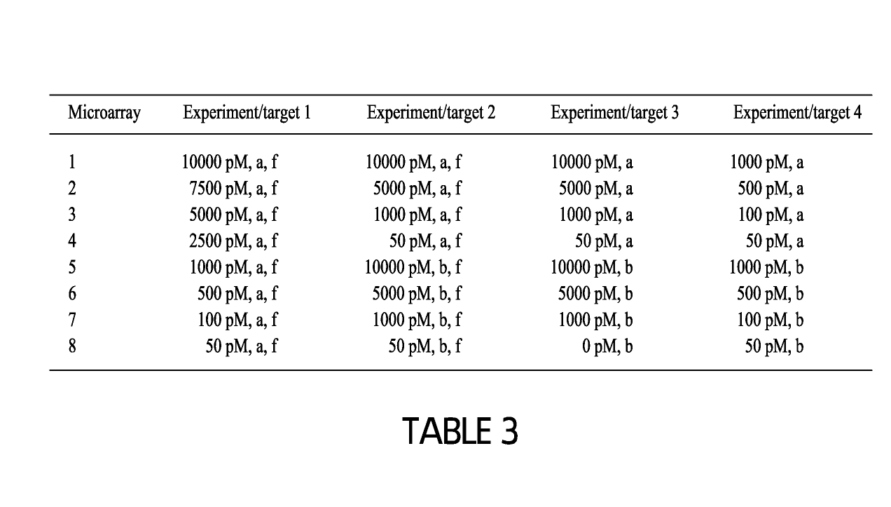

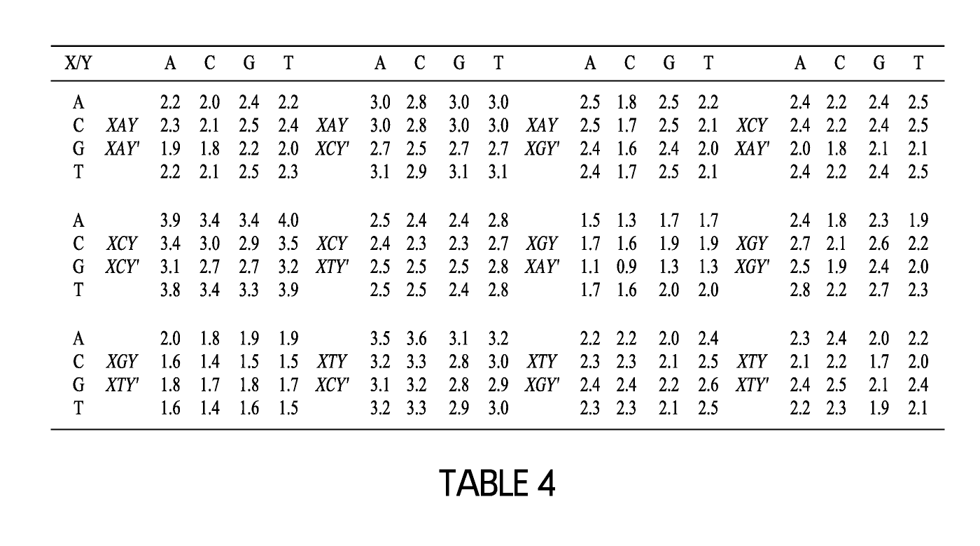

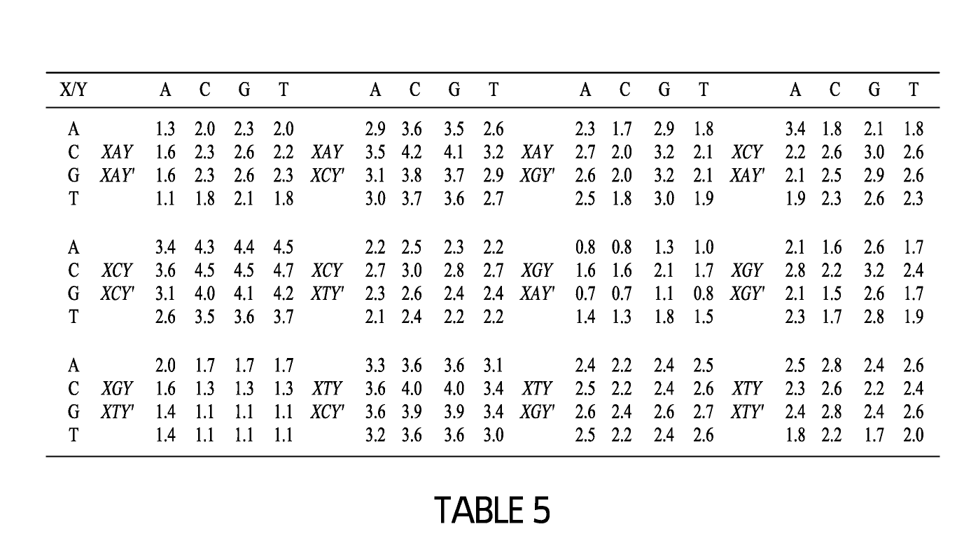

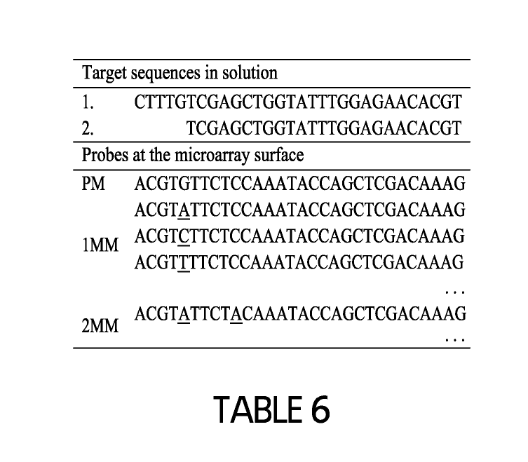

Algorithm. FIG. 16 shows a flowchart of the algorithm used for the detection and identification, suitable for the different embodiments presented herein. The algorithm consists of a central loop that generates iteratively sequences . . . tₙ₋₁tₙ, tₙ₊₁. . . (where n is the iteration step), which are successive in silico hypotheses about the composition of the sample. The loop is repeated until convergence is reached. Two types of convergence are obtained. In some cases, after a certain number of iterations, the algorithm shows a collapsed I-ΔΔG plot similar to that seen in FIG. 3A. This means that a unique target nucleic acid is present in the sample. In this case, the algorithm ends at the block (b) of the flowchart and returns the output tₙ as the sequence composition of the sample. In other cases, the algorithm converges to a two-cycle state i.e. tₙ=tₙ₊₂=tₙ₊₄=. . . and tₙ₊₁=tₙ₊₃= . . . where the I-ΔΔG plots are always branched (similar to that seen in FIG. 3B). This two cycle state is a signature for a sample composed by a mixture of two target nucleic acids tₙ and tₙ₊₁. In this case, the algorithm ends at the block (e) and returns the aforementioned two sequences as output.

An important part for generating new in silico hypotheses is the decision block (a) of the flowchart. At this point, the algorithm checks if the I-ΔΔG plot is collapsed or branched. In the latter case, a new in silico hypothesis tₙ₊₁ is generated. To understand how this is done, the origin of the branching has to be elucidated in some detail. The plot of FIG. 3B is obtained by calculating ΔΔG using a wrong in silico hypothesis. For instance, if the actual sequence in solution and the hypothesis sequence made for the ΔΔG calculation differ in a single nucleotide (e.g. actual target=T; hypothetical target=G), when considering the probes with an A at the different nucleotide position, the in silico hypothesis estimates the ΔΔG as an ΔG mismatch, resulting in overestimating the ΔΔG. Likewise, for the probes with a nucleotide C, the ΔΔG is underestimated. Thus, the splitting into different branches is due to the wrong estimates of free energy for each probe in the position where actual target and in silico hypothesis differ. It is important to notice here that the probes in the different branches systematically differ from each other by specific nucleotides at specific locations. This systematic sequence deviation will be used to decide whether the I-ΔΔG plot is branched or not. The correct hypothesis can readily be constructed by selecting out the top left branch, determining the systematic sequence deviation (nucleotide position and type) in this probe subset, and implementing this nucleotide change in the previous hypothesis. This is precisely how a new in silico hypothesis denoted by tₙ₊₁ is generated in block (c) by the algorithm. In principle, for a unique sequence sample, one can start with any hypothesis and iterate until a collapsed I-ΔΔG plot is found. However, when the sample is a mixture of two target sequences, the plots will always have branches, as over- and under-estimation of the data during the ΔΔG calculation will always occur. For each iteration, a new hypothesis is generated and when the tₙ₊₁ sequence is equal to the tₙ₋₁ sequence as evaluated in block (d), the algorithm is stopped and gives as output a mixed sample composed by sequences tₙ and tₙ₊₁.

Decoding of Coded Samples.

The algorithm was tested on seven coded clinical samples selected by Janssen Diagnostics out of a repository of samples for which the Sanger sequencing had been performed. The proposed method decoded the samples successfully. We here briefly discuss the working of algorithm.

Generally, the initial hypothesis tᵢ corresponded to the sequence with highest clinical frequency. However, the algorithm also converged to the same results when t₁ is one of the other 99 sequences of the PM set.

Typically, the I-ΔΔG plot produced from the initial hypothesis t₁ shows that in this first iteration the data are very scattered. First, a set of data points is selected, which deviate the most from the expected thermodynamic behavior (i.e. Equation [3]). The nucleotide composition of the corresponding probes is analysed, e.g. via a histogram of mismatches per position with respect to the initial hypothesis t₁. In the algorithm, a threshold value of 70% is chosen as the minimum limit for the fraction of common mismatches per base positions between the selected probes and the current hypothesis. Based on this threshold, the selected probes are considered mismatching against the hypothesis t1 at a number of specific base positions by specific nucleotides. This information is used to generate a new hypothesis t₂ by swapping the corresponding nucleotides on hypothesis t₁ so it becomes complementary to the specific nucleotides detected in the mismatching base positions. The next iteration is the calculation of the I-ΔΔG plot from t₂. If the data is still scattered, the procedure is repeated: the most deviating set of points is analysed and the non-matching nucleotide(s) is (are) identified, and a subsequent in silico hypothesis t₃ is then obtained. Again, the I-ΔΔG plot is calculated, now based on t₃.

If the data is no longer scattered, or the analysis of the most deviating set of points does not provide a strong signature for any common mismatch (<70%), the algorithm concludes that the sample contains a unique sequence t₃.

If on the other hand, the sample contains a mixture of two sequences, the I-ΔΔG plot will always deviate from the predetermined correlation (i.e. Equation [3]). In this case, the analysis of the most deviating points generate a cycle in which sequences tₙ and tₙ₊₂ are identical, indicating that the sample is actually a mixture of two target nucleic acids, with sequences tₙ and tₙ₊₁,

Resolving ambiguity from two degenerate bases. Due to uncertainties that are inherent in the Sanger method, this method gives rise to degenerate nucleotides in the obtained sequences. The letters R and Y denote the degenerate bases A or G (purines) and C or T (pyrimidines), respectively. These uncertainties are caused e.g. by the presence of mixtures of two sequences in a given clinical sample. In the case of a single degenerate nucleotide, the sample is identified as a unique mixture. However, two different types of mixtures are possible in the case of two degenerate nucleotides in the same sequence. For instance a degenerate RR pair can mean either a AA/GG mixture or a AG/GA mixture. Advantageously, the analysis of the hybridization data from the microarray experiments allows to resolve this ambiguity because the hybridization free energies for each case are different.

The importance of this information lies in its clinical relevance, for example, a correct identification of the mutations RTR occur in codon 184 gives an idea about the stage of resistance. This is interesting because during treatment with lamivudine, initially isoleucine mutants are present, which are subsequently replaced by valine variants.

In conclusion, based on hybridization thermodynamics sequence information could be obtained from a fragment of the HIV-1 RT gene. In the HIV example considered here, due to the high mutation rate of the virus, the fragment analysed can occur in more than a hundred different variants. In addition, one needs to distinguish between samples composed by a single sequence (unique) from samples in which two or more different sequences coexist (mixed). For clinical purposes, it is important to identify early enough the rising of a resistant strain. Our microarray analysis is based on a large number of measured intensities not just from perfectly matching probes but also mismatching probes. Although in general the effect of the hybridization of a very low abundance sequence in a mixed sample can be small for each individual intensities, the correlated effect on a large number of different probes can be detected even from a target sequence at relatively low abundance (about 1%). An iterative algorithm is presented that successively generates in silico hypotheses for the sample composition and checks them against thermodynamic models. The algorithm identified correctly the sequences in the sample.

### Example 2

### Theoretical Considerations

By way of illustration, the present invention not being limited thereby, an example is given on how the free energy parameters of the nearest neighbor model could be fitted to results obtained for hybridization in DNA microarrays. The example illustrates features and advantages of embodiments according to the present invention. Whereas for the present example theoretical considerations are taken into account, embodiments of the present invention are not limited thereby. For the present study several hybridization experiments were performed, each with a single oligonucleotide sequence (referred to as the target of interest herein) in solution at different concentrations. Four different targets were used in the experiments, and their sequences are given in Table 1. The sequences contain a 30-mer hybridizing stretch followed by a 20-mer poly(A) spacer and a Cy3 label at the 3′-end of the sequence. Each target oligo was bought in duplicate in order to check the quality of the target synthesis. Reference will be made to the two duplicated oligos as a and b. The sequences printed at the microarray surfaces and referred here as the probes were chosen to contain up to two mismatches, following the scheme shown in Table 2. Mismatches were inserted from nucleotides 6 to nucleotide 25 along the 30-mer sequences in order to avoid terminal regions. In the probes with two mismatches these were separated by at least 5 nt. Given the nucleotide of the target strand there are three different possible mismatching nucleotides and 20 available positions, hence in total 60 single mismatch sequences. A similar counting for double mismatches yields 945 different sequences (Table 2). The total number of probe sequences, including the perfect matching one, is 1006. For each experiment one target and one 8×15K custom Agilent slide was used. This slide consists of eight identical microarrays and each of these can contain up to more than 15000 spots. The 1006 probe sequences were spotted in the custom array 15 times: in 12 replicates a 30-mer poly(A) was added on the 3′-side (surface side), in order to assess the effect of a sequence spacer. Three replicates contained no poly(A) spacer. The eight microarrays of one slide have to be hybridized during the same experiment, but a different target solution can be used. In the experiments, the target concentrations ranged from 50 to 10000 pM according to the scheme given in Table 3. In Experiment 1 only target a was used, while in the Experiments 2, 3 and 4 both replicated targets (a and b) were used. Finally, in Experiments 1 and 2 a fragmentation of the target was performed before hybridization (see section on hybridization protocol for details). The four 30-mer target sequences were selected from fragments of human genes having a GC content ranging from 43% to 50%. A criterion for selecting the target sequences was the requirement that the probes constructed following the scheme in Table 2 would yield a roughly flat histogram of mismatch types, so that all mismatches are approximately equally present in the experiments.

For the experiments, the commercially available Agilent platform was used and a standard protocol with Agilent products was followed, as described subsequently. The target oligonucleotides were OliGold© from Eurogentec, Seraing, Belgium. Hybridization mixtures contained one target oligonucleotide with a 3′-Cy3 end labeling diluted in nuclease-free water to the final concentration together with 5 μl 10× blocking agent and 25 μl 2× GEx hybridization buffer HI-RPM. In Experiments 1 and 2 the addition of the hybridization buffer was preceded by a fragmentation step, 1 μl fragmentation buffer was added followed by an incubation of 30 min at 60° C. This fragmentation buffer is customarily used in Agilent hybridization platforms and produces target sequences of reduced length in order to speed up the hybridization reaction. Too long sequences, as obtained from biological extracts, e.g. from reverse transcription of mRNA samples, have a reduced hybridization efficiency due to steric hindrance. By comparing experiments with and without fragmentation, it was found that the fragmentation step has little effect on the results. The hybridization mixture was centrifuged at 13000 r.p.m. for 1 min and each microarray of the 8×15K custom Agilent slides was loaded with 40 μl.

The hybridization occurred in an Agilent oven at 65° C. for 17 h with rotor setting 10 and the washing was performed according to the manufacturer's instructions. The arrays were scanned on an Agilent scanner (G2565BA) at 5 mm resolution, high and low laser intensity and further processed using Agilent Feature Extraction Software (GE1 v5 95 Feb07) that performs automatic gridding, intensity measurement, background subtraction and quality checks.

In the present example, use is made of the Langmuir model for describing the dynamics of hybridization by a rate equation for 0, the fraction of hybridized probes from a spot as follows

$$\frac{d θ}{dt} = {ck}_{1} ( 1 - θ ) - {k}_{- 1} θ \qquad [ 4 ]$$

where c is the target concentration and k₁ and k₋₁ are the attachment and detachment rates. The equilibrium value for θ can be obtained from the condition dθ_{eq}/dt=0. Using the link between the rates and equilibrium constants, i.e. k₁/k₋₁=e^{-ΔG/RT}, with ΔG the hybridization free energy, R the gas constant and T the temperature one finds

$${θ}_{eq} = \frac{{ce}^{- Δ G / RT}}{1 + {ce}^{- Δ G / RT}} \qquad [ 5 ]$$

which is the so-called Langmuir isotherm. To link this isotherm to the measured quantities one assumes that the fraction of hybridized probes is linearly related to the measured fluorescent intensity measured from a spot, which yields

$$I = \frac{{Ace}^{- Δ G / RT}}{1 + {ce}^{- Δ G / RT}} \qquad [ 6 ]$$

Here I is the background-subtracted intensity, where the background subtraction, as explained above is done by Agilent Feature Extraction software. Where reference is made to the intensities, these are intensities that are background subtracted. A is a constant which is an overall scale factor.

Far from chemical saturation, i.e. when only a small fraction of surface sequences is hybridized (i.e. c e^{-ΔG/RT}<<1) one can neglect the denominator in Equation [5] to get:

I≈Ace^{-ΔG/RT} [7]

In the nearest neighbor model, the hybridization free energy of perfect complementary strands is approximated as a sum of dinucleotide terms. For instance

$$Δ G ( \begin{matrix}ATCCT \\ TAGGA\end{matrix} ) = Δ G ( \begin{matrix}AT \\ TA\end{matrix} ) + Δ G ( \begin{matrix}TC \\ AG\end{matrix} ) + Δ G ( \begin{matrix}CC \\ GG\end{matrix} ) + Δ G ( \begin{matrix}CT \\ GA\end{matrix} ) + Δ {G}_{init} \qquad [ 8 ]$$

where ΔGᵢₙᵢₜ is an initiation parameter. Since only differences of ΔG between a perfect matching hybridization and a hybridization with one or multiple mismatches [Equation [10]] are considered, this initiation parameter will not contribute and it is omitted further in this example. For DNA/DNA hybrids, symmetries reduce the number of independent parameters to 10. The nearest neighbor model can be extended to include single internal mismatches; as an example it is considered that the free energy of a stretch with an internal mismatch of CT type

$$Δ G ( \begin{matrix}AT \underline{C} CT \\ TA \underline{T} GA\end{matrix} ) = Δ G ( \begin{matrix}AT \\ TA\end{matrix} ) + Δ G ( \begin{matrix}T \underline{C} \\ A \underline{T}\end{matrix} ) + Δ G ( \begin{matrix}G \underline{T} \\ C \underline{C}\end{matrix} ) + Δ G ( \begin{matrix}CT \\ GA\end{matrix} ) \qquad [ 9 ]$$

The mismatching nucleotides are underlined and for notational reasons the mismatch is always put in the second part of the dinucleotide (which requires the use of symmetry like here in dinucleotide term three). There are 12 types of mismatches and 4 types of flanking nucleotide pairs, hence in total there are 48 mismatch parameters of dinucleotide type. There are several possible ways of extracting the 48+10 dinucleotide parameters from the experimental data.

One can either fit the full Langmuir isotherm [Equation [5]], or for experiments at sufficiently low concentrations one could consider the limiting case of Equation [7]. In addition, the parameters could be extracted either from an experiment at fixed concentration c, by comparing the intensities of different probe sequences, or from experiments at different concentrations by analyzing the intensities of identical probe sequences over a concentration range. Focus is put on the low concentration data and use of Equation [7] for the analysis at fixed c. Equation [7] contains the constant A which is an overall scale factor relating the hybridization probability to the actual measured fluorescence intensity. This quantity may fluctuate from experiment to experiment. For instance, the optical scanning influences A, as this is proportional to the laser intensity used. Also hybridizations in different slides might occur at slightly varying conditions and there can be small differences in the manufacturing of the slides. Focus will be put on relative intensities and relative free energies, i.e. for each microarray the perfect match of that microarray will be used as a point of reference. The logarithmic ratios of the intensities with the perfect match intensity are denoted as

$${y}_{i} = ln {I}_{i} - ln {I}_{PM} = - \frac{Δ G - {ΔG}_{PM}}{RT} = - \frac{ΔΔ G}{RT} \qquad [ 10 ]$$

for which the exact value of A is irrelevant and only the relative free energy differences ΔΔG (which is for each probe a positive number) are considered. In ΔΔG of a duplex, only dinucleotide parameters which are flanking a mismatch remain, the other parameters cancel out in the subtraction. For example from Equations [8] and [9] one gets

$$ΔΔ G ( \begin{matrix}AT \underline{C} CT \\ TA \underline{T} GA\end{matrix} ) = - Δ G ( \begin{matrix}T \underline{C} \\ A \underline{T}\end{matrix} ) + Δ G ( \begin{matrix}G \underline{T} \\ C \underline{C}\end{matrix} ) - Δ G ( \begin{matrix}TA \\ AT\end{matrix} ) - Δ G ( \begin{matrix}AC \\ TG\end{matrix} ) \qquad [ 11 ]$$

In this equation the lower strand refers to the target sequence in solution, which is fixed. The upper strand is that of the probe sequence attached to the solid surface. Hence, the ΔΔG of a duplex with one mismatch can be written as a sum of two mismatch dinucleotide parameters minus two matching dinucleotide parameters. As it is assumed that the nearest neighbor model is valid, the same reasoning can be applied to duplexes with two mismatches which results in a sum of four mismatch dinucleotide parameters minus four matching dinucleotide parameters. The model can now be written as

$${y}_{i} = {\sum }_{α = 1}^{58} {X}_{i α} \frac{Δ {G}_{α}}{RT} \qquad [ 12 ]$$

where a is the index running over the 58 possible dinucleotide parameters and X is a frequency matrix, whose elements X_{iα} are the number of times the dinucleotide parameter a enters in ΔΔG of probe sequence i. With a simple extension of matrices and vectors one can rewrite the problem as

\vec{y}=X\vec{ω} [13]

Where it is defined that

ω_{α}=ΔG_{α}/RT. Having written the problem in Equation [13] as a linear one, one can now apply the standard approach to find the optimal values of the parameters. The procedure consists in minimizing S=(\vec{y}−X\vec{ω})² which amounts to solving the following linear equation

X^{T}(\vec{y}−X\vec{ω})=0 [14]

where X^{T} is the transpose of X.

To obtain \vec{ω} from Equation [14] one has to invert the 58 x 58 matrix X^{T}X. In the case that X^{T}X is not invertible one applies a singular value decomposition. In the present case the matrix is not invertible. Zero eigenvalues of the matrix XTX come from re-parametrizations that leave the physically accessible parameters ΔΔG invariant. The dinucleotide mismatch parameters are not uniquely determined, as these parameters are entering in the expression for the total ΔG in pairs [Equation (9)]. For instance, a re-parametrization of the type:

$$Δ {G}^{′} ( \begin{matrix}x & \underline{C} \\ {x}^{′} & \underline{T}\end{matrix} ) = Δ G ( \begin{matrix}x & \underline{C} \\ {x}^{′} & \underline{T}\end{matrix} ) + ɛ \\ Δ {G}^{′} ( \begin{matrix}y & \underline{T} \\ {y}^{′} & \underline{C}\end{matrix} ) = Δ G ( \begin{matrix}y & \underline{T} \\ {y}^{′} & \underline{C}\end{matrix} ) - ɛ \qquad [ 15 ]$$

for every pair of complementary nucleotides x; x′ and y; y′ leaves the total ΔG invariant, as it can be verified directly from Equation [9]. Similar re-parametrizations are possible for mismatches of type AG, AC and TG. Next to these there are three invariances of ΔΔG that involve a re-parametrization of both mismatch and matching dinucleotide parameters. Hence one has at least seven zero eigenvalues in X^{T}X.

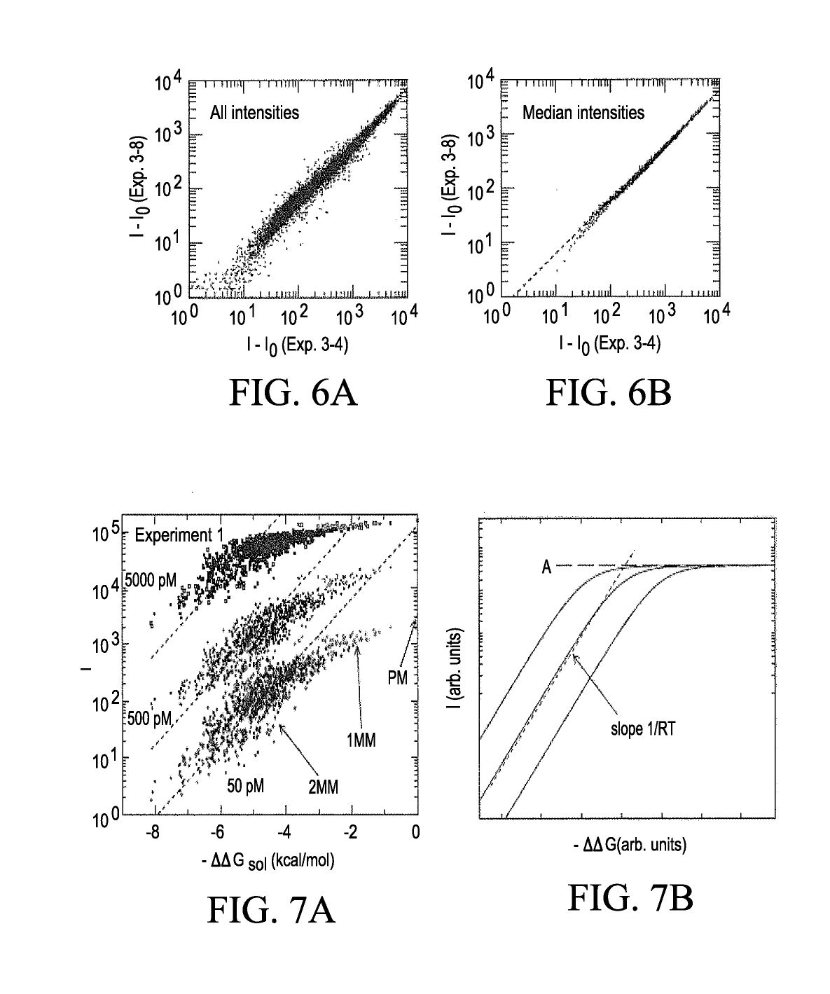

As a control of the reproducibility of the result, the intensities correlation between analogous spots in replicated experiments is considered. The replicated hybridizations were carried out on two microarrays of the same slide, with two identical but separately synthesized and labeled target oligos, at the same manually prepared concentration in solution Table 3. FIGS. 6A and 6B is an example thereof. It shows correlation plots between two replicated hybridizations. Two plots are shown, one with the full 15K intensities (FIG. 6A) and one in which the median of the intensities of the 15 replicated spots are taken (FIG. 6B). In the former some data spreading is observed, which is greatly reduced when the median over 15 replicated spots is taken. Note that the experimental data do not align perfectly on the diagonal of the graph, this may be attributed to the manual preparation of the solutions or to differences in the oligos (synthesis or labeling). Data from different microarrays are aligned on a line of slope equal to one in the log-log plots of FIGS. 6A and 6B, which implies a linear relationship between the intensities. In general, replicates show a strong

correlation between median intensities, which is an indication of a good reproducibility of the results. In this median the probes with and without poly(A) spacer are included. No significant difference was found in the intensities from spots with poly(A) and without poly(A) spacer. From this point on, the median intensity of 15 replicates is always used and simply referred to as the intensity of a probe, and because of the good reproducibility only the data produced by hybridizations with oligo synthesis a (Table 3) will be discussed. Next, the relation between the intensities and the corresponding ΔGₛₒₗ for hybridizations in solution with one or two mismatches are considered. In the case of two mismatches ΔGₛₒₗ was calculated as the sum of nearest neighbor parameters for individual mismatches, assuming that the presence of two mismatches does not involve additional terms in the free energy, i.e. they do not interact. In the experiment the minimal distance between two mismatches is 5 nt, which is considered sufficient, in first approximation to support the non-interaction assumption. In the calculation of ΔG from the tabulated values of ΔH and ΔS the temperature was set to the experimental value T=65° C.

FIG. 7A shows the plot of the intensities versus −ΔΔGₛₒₗ as taken from the nearest neighbor model with the existing tabulated values for hybridization in solution. ΔΔGₛₒₗ is obtained by subtracting from all free energies that of the PM sequence, which is taken as a reference. As a consequence, for the PM intensities ΔΔGₛₒₗ=0. Each plot in FIGS. 7A and 7B contain 1006 data points obtained from the median value of the 15 replicated spots on each array.

As it is well-known from several studies of melting/hybridization in aqueous solution, the hybridization free energy ΔGₛₒₗ depends on the buffer conditions, and in particular of the ionic strength of the solution. Particularly studied was the effect of salt concentration (NaCl), which is usually assumed to be independent of sequence, but to be dependent on oligonucleotide length. Melting experiments in solution are consistent with the following dependence on Na ion concentration

ΔGₛₒₗ=ΔGₛₒₗ(1M[Na⁺])−αNln[Na⁺] [16]

Where ΔGₛₒₗ (1M[Na+]) is measured at 1M NaCl, N is the number of phosphates in the sequence and a is a constant.

Salt has mostly an effect on interactions with the negatively charged phosphate molecules. It is hence plausible to expect the same type of correction as Equation [16] also for sequences carrying mismatches. If that is the case, the salt dependence cancels out from ΔΔGₛₒₗ, which is the quantity of interest. Therefore the value will be set at 1M NaCl in ΔGₛₒₗ.

FIG. 7A shows the data for Experiment 1 at three different concentrations, from bottom to top of 50, 500 and 5000 pM. When plotted as functions of −ΔΔGₛₒₗ, the data points tend to cluster along single monotonic curves. This already suggests a fair degree of correlation between ΔGₛₒₗ and ΔG_{μarray}. The experiment at 5000 pM shows a pronounced saturation of the intensities, as expected from the Langmuir model [Equation [5]]. Sufficiently far from saturation one expects a linear relationship between the logarithm of the intensity and ΔG, as given by Equation [7]. FIGS. 7A and 7B show that the low concentration data at low intensities follow approximately a straight line with the slope 1/RT expected from equilibrium thermodynamics at T=65° C., which is the experimental temperature.

However, the global behavior of the three concentrations is at odds with the Langmuir model, which predicts that intensity versus free energy plots for different concentrations should saturate at a common intensity value A, as indicated in FIG. 7B. Although one may expect some variations on A from experiment to experiment, the data of FIG. 7A are hard to reconcile with the Langmuir model. It is concluded that the hybridization data deviate from the full Langmuir model of Equation [5], but they are in rather good agreement with its limiting low intensities behavior [Equation [7]]. In order to obtain estimates of the free energies ΔΔG_{μarray} from microarray data, then Equation [67 will be used and restriction will be made to the lower concentration data.

To fit the 58 parameters of the nearest neighbor model the lowest concentration data are used, i.e. 50 pM. Hereto the algebraic procedure explained above is applied, which fits the logarithm of the ratios I/I_{PM} and which assumes that the data can be described by Equation [10]. For low concentrations this assumption is expected to be correct for the lower intensities but not for the highest intensities, which deviate from the Langmuir isotherm as shown in FIGS. 7A and 7B. This poses a problem for the fitting procedure since it was designed with the perfect match intensity IPM as a reference [Equation [10]]. One may think to circumvent this problem by restricting the fit to low intensities, for instance only to probes with two mismatches and rewrite Equation [10] using as reference not IPM, but for instance one of the intensities of a probe with two internal mismatches. This procedure turns out to be of little practical use for the purpose of estimating the free energy difference between perfect matching sequences and sequences with one or multiple mismatches and for which the PM reference value is necessary.

From the analysis of plots of intensity versus ΔΔGₛₒₗ (FIGS. 7A and 7B), one finds that the PM intensity is systematically lower than that predicted by Equation [7], which is the straight line in FIG. 7A. Hence, the relative intensities I/I_{PM} of the probes that contain mismatches are systematically higher than those predicted by Equation [7].

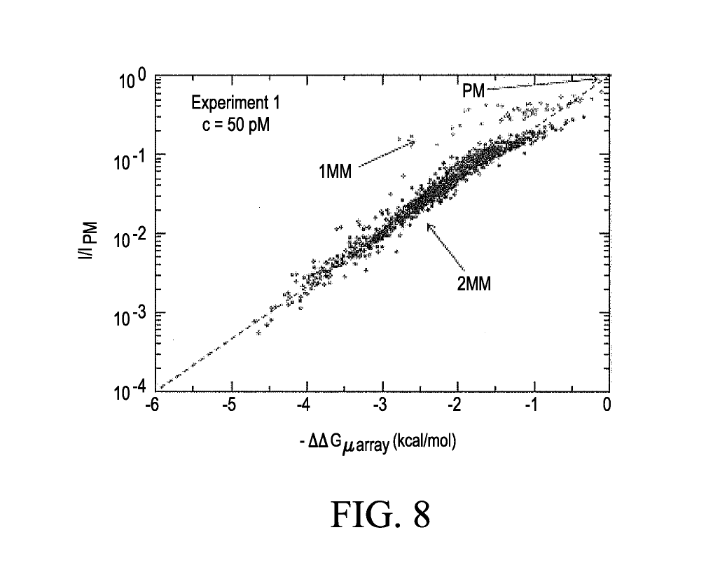

Consequently, a direct fit of the experimental data to Equation [10] underestimates the effect of a mismatch, which will result in free-energy penalties that are too small. The result of the fit is shown in FIG. 8. One can notice that the ΔΔG range is indeed smaller than the one from hybridization in solution (FIGS. 7A and 7B).

Moreover, the underestimation of ΔΔG is more severe for probes with two mismatches than for those with only one, since ΔΔG is a sum of contributions per mismatch. This produces a discontinuity of the curve from double to single mismatches. The appearance of this discontinuity is another evidence of the fact that Equation [7] is not valid in the full range of intensities.

In order to solve this problem, one would need to fit the data with a more general model I(c, ΔG) that incorporates the observed deviations from Equation [7]. Moreover, the choice of this model may considerably influence the fitted nearest neighbor parameters. A safer compromise is to start from the observation that Equation [7] is followed by the large majority of the low concentration data points in FIGS. 7A and 7B. Hence a fit to the low concentration limit of the Langmuir model seems reasonable. Unfortunately, one of the points deviating from Equation [7] is the PM intensity, which is used as reference measure. In order to calibrate the fit correctly one should reweight the reference PM intensity. The data therefore were fitted against Equation [10] using instead of the actual PM intensity as a reference, a rescaled value I*_{PM}=αI_{PM}, which is the value the PM intensity would have if the data would agree with Equation [7] in the whole intensity range. α is estimated from the crossing of the 50 pM fitting line in FIG. 7A with the ΔΔG=0 axis. This estimate is α=30.

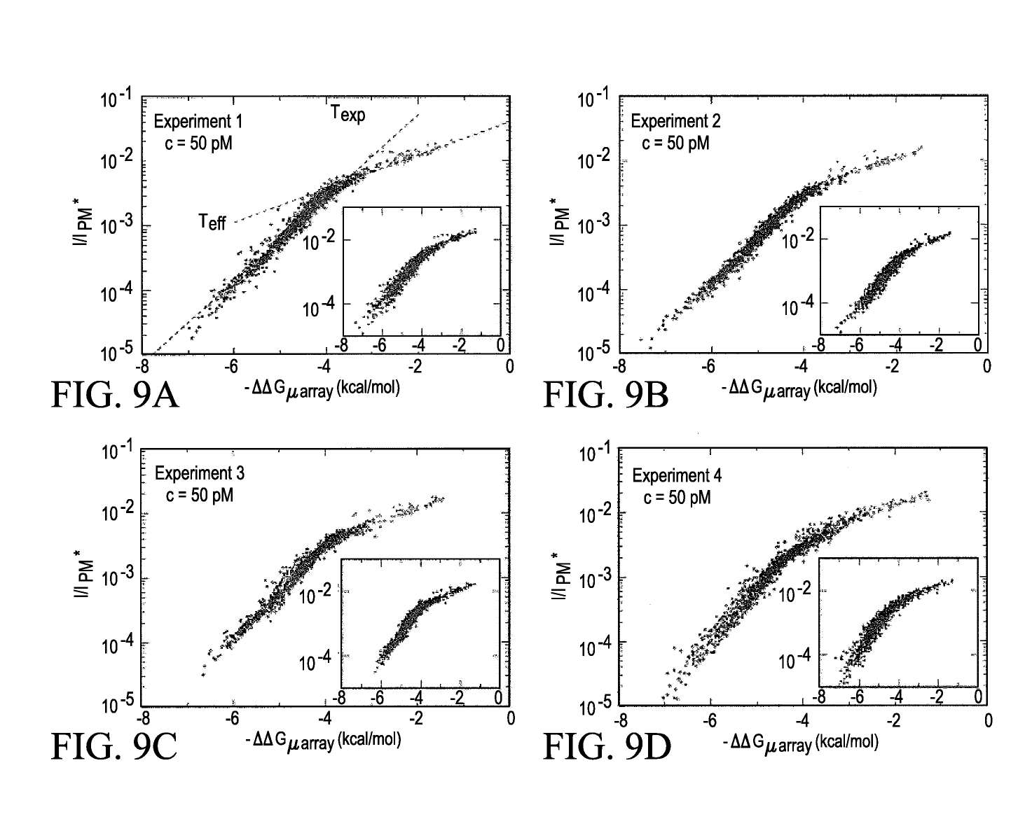

The effects of a change in a on the fitting parameters will be discussed below. FIGS. 9A-9D show the result of the fit to Equation [10], using α=30. In the main frames each experiment is fitted independently. In the insets, the free-energy parameters are obtained from a simultaneous fit of all 50 pM experiments.

The latter data produce more accurate parameters, as they come from using four independent experiments (the four experiments at 50 pM, oligo synthesis a, in Table 3), hence the 58 parameters are obtained on sampling over 1006×4 data points. Both the free-energy range and the continuity of the curves in FIGS. 9A-9D are now as expected. The data show very little spreading in comparison with the curves in FIG. 7A. A quantification of the spreading for a monotonic curve can be assessed by the Spearman's rank correlation coefficient, which for all four experiments is very close to 1. This is an indicator of the reliability of the nearest neighbor fitted parameters. The ratio of data points over tuning parameters is as large as 4024/58, which ought to yield a reliable fit. Moreover, although the data are fitted to a linear model, all four experiments show a clear deviation for the highest intensities.

This is an indication against overfitting, which would result in a fully linear curve with erroneous fitting parameters. Therefore, it is concluded that the deviations from the Langmuir isotherm observed in all four experiments is a robust feature of the system and that the resulting free energy parameters are physically meaningful. It is also verified that the free energy parameters obtained from the fit are quite stable whether one fits the whole set of experimental data, or whether the fit is restricted to the lowest intensity scales (e.g. I/I*_{PM}≤5×10⁻³) where all data clearly follow Equation [7]. This is because the large majority of experimental points in FIGS. 9A-9D are located in the lowest intensity scales, anyhow. Hence, this additional data filtering has little effects on the parameters.

Table 4 shows the free energy parameters ΔΔG_{μarray} as obtained from the above fitting procedure. Because of the degeneracies mentioned above, the dinucleotide parameters are not uniquely determined. Triplet parameters are, however, unique, and these are given in the table. The ΔΔG for triplets are defined, for instance

$$ΔΔ G ( \begin{matrix}A \underline{C} G \\ T \underline{T} C\end{matrix} ) = Δ G ( \begin{matrix}A \underline{C} G \\ T \underline{T} C\end{matrix} ) - Δ G ( \begin{matrix}AAG \\ TTC\end{matrix} ) \qquad [ 17 ]$$

where the upper strand is 5′-3′ oriented. The lower strand is the invariant target sequence, the upper strand is the probe sequence. Hence the ΔΔG parameters are measured subtracting the reference perfect match probe. Note that because of this subtraction one has

$$ΔΔ G ( \begin{matrix}A \underline{C} G \\ T \underline{T} C\end{matrix} ) \neq ΔΔ G ( \begin{matrix}C \underline{T} T \\ G \underline{C} A\end{matrix} ) \qquad [ 18 ]$$

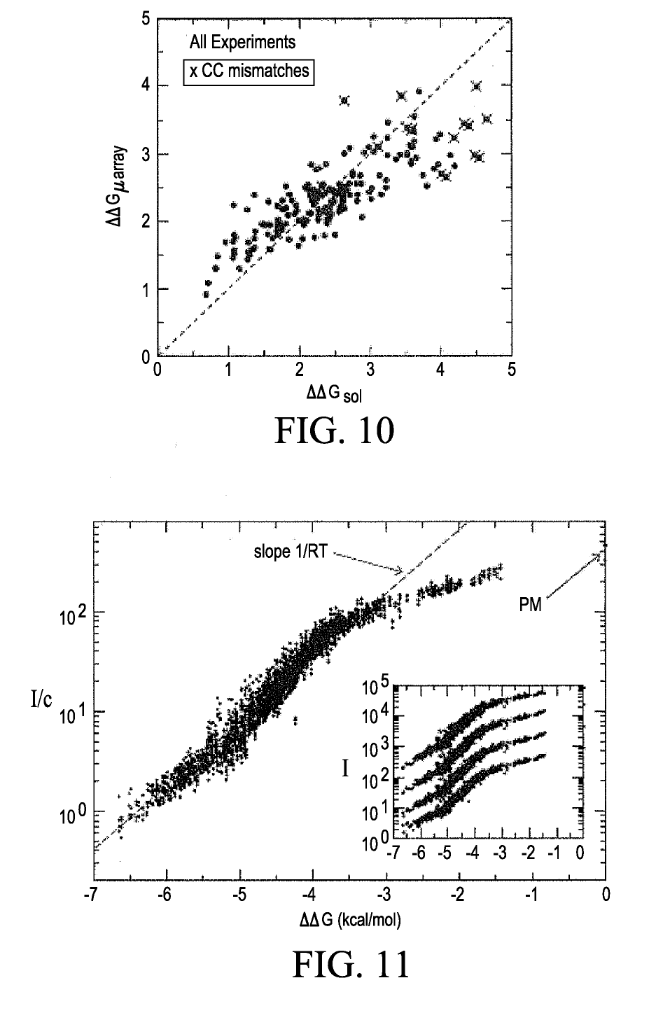

as the reference PM sequence is different in the two cases. Using standard linear regression tools, the error bar was estimated on the parameters of Table 4 to be equal to 0.2. In Table 5 the ΔΔGₛₒₗ for triplets following the same notation as in Table 4 are presented. As mentioned before the data in solution are at T=65° C. and 1M [Na]. FIG. 10 shows a plot of the two free energies ΔΔG_{μarray} versus ΔΔGₛₒₗ. A clear quantitative correlation between the two is observed. The Pearson correlation coefficient is 0.839. In comparing the two sets, it can be noted that the 16 mismatches of CC appear to be the most deviating in the two cases.

As discussed above, the fit was done with a re-scaled PM intensity, using a factor α=30. The analysis was repeated for other values of α. Varying α causes a global shift of the data in Table 4 by an a-dependent constant.

This shift does not affect the slope or correlation of the data in FIG. 10. By using α=50 a positive shift of 0.17 was found, while setting α=20 produces a shift of −0.14. These two values of a are our estimate of the largest range of variability for this parameter. In general, the procedure of re-weighting the PM intensity with a introduces a global error ±0.2 affecting all parameters in Table 4.

One of the advantages of the experimental setup chosen in this work is that one can obtain in principle all parameters in a single experiment, as all hybridization reactions with one or two mismatches occur in ‘parallel’ on a single array. However, a drawback is that in this setup one can determine only the free energy and not the contribution of enthalpy and entropy separately, which would allow to extend the parameters to other temperatures.

In the above example, focus was put on the determination of ΔΔG which is the free energy difference between a perfect matching hybridization and an hybridization where the probe sequences have one or more internal mismatches. Quantifying the effect of internal mismatches is important for a better understanding of cross-hybridization effect, which is the unintended binding of non-perfectly complementary sequences to a given probe. Moreover, this understanding could have some practical consequences for optimal probe design. An advantage of the parameter ΔΔG is that it is insensitive to the free-energy initiation parameter [Equation [8]] and the scaling factor A [Equation [5]] and [7] and that it is expected to be less sensitive to buffer conditions as ionic salt etc. The example, showing custom Agilent arrays shows that there is a strong correlation, also on the quantitative scale, between ΔΔGₛₒₗ and ΔΔG_{μarray}. This correlation is shown in FIG. 10 with explicit free-energy values given in Table 4 and 5. A fit of the interaction parameters from microarray data shows a much better agreement of the data with the thermodynamic models (compare FIGS. 7A and 7B with FIG. 8). However, in the absence of dedicated experiments for the determination of interaction free energies on a DNA microarray, the results of this work suggest that one could use the corresponding hybridization free energies in solution as approximations for them.

As a correlation between ΔGₛₒₗ and ΔG_{μarray} has by now been observed in several different microarray platforms, it is fair to expect that such a correlation is a general feature of microarrays.

It is interesting to remark that the deviation from the Langmuir model ‘enhances’ the cross-hybridization problem because there is a smaller effect on intensity for a given free energy penalty (smaller slope in FIGS. 9A-9D). As an example, a mismatch with ΔΔG=2.5 kcal/mol (a typical value from Table 4) corresponds to a I/I_{PM} ratio of≈0.02 in the regime governed by the Langmuir model, compared to≈0.2 in the deviating regime. This implies that in the deviating regime a significant fraction of the amount of target binding to a PM probe binds to a probe carrying one internal mismatch.

In a second example, hybridization in DNA microarrays is discussed as also discussed in the first example. The second example illustrates the existence of slow relaxation phenomena for hybridization in DNA microarrays.

Experiments are described wherein hybridization takes place between the surface-bound sequences (referred to as probes) and the sequences in solution (targets) carrying a fluorophore. The amount of hybridized target is measured from the emitted fluorescence from a given location (spot) on the microarray surface. It is illustrated that, contrary to a widespread belief, in DNA microarrays relaxation times may largely exceed typical experimental times, causing a breakdown of thermal equilibrium. Experiments are performed on a commercial microarray platform under the same buffer conditions as in typical biological experiments. They are further corroborated by the analysis of an extended kinetic model. In equilibrium one expects that the intensity measured from a spot is described by the Langmuir model

$$I = \frac{{Ace}^{\frac{Δ G}{RT}}}{1 + {ce}^{\frac{Δ G}{RT}}} \approx {Ace}^{\frac{Δ G}{RT}} \qquad [ 19 ]$$

where A sets the intensity scale, R is the gas constant, T is the temperature, c the target concentration and ΔG the hybridization free energy. It is to be noticed that a different sign convention is used compared to the first example. This is merely a matter of choosing the bound or the unbound state as the reference state for free energy differences. In Eq. [19] it took ce^{ΔG/RT}<<1 (weak binding and small concentrations), a limit which applies to the experiments discussed here.

The experimental setup is schematically shown in Table 6 and further similar to the first example.

FIG. 11 shows a plot of I/c vs. ΔΔG for four experiments at different concentrations using the setup of Table 6 (the target sequence is the 30-mer). ΔΔG≡ΔG−ΔG_{PM} is the difference in hybridization free energies between a given sequence and the perfect match (PM) sequence, calculated from the nearest-neighbor parameters. The collapse of the I/c vs. ΔΔG plots into a single curve shows that/∝ c as expected from the low concentration limit of Eq. [18].

However, the dependence on ΔG is not in full agreement with Eq. [19]. In the regime deviating from Eq. [19] log I scales approximately linearly with ΔG but with a slope smaller than 1/RT.

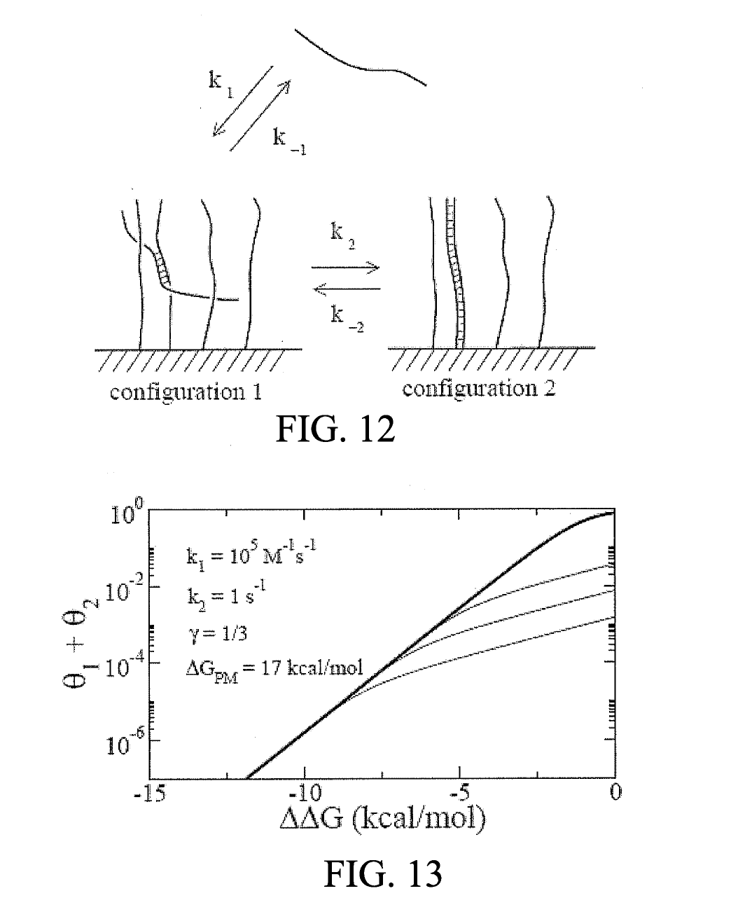

Hybridization dynamics of oligonucleotides in aqueous solution is usually described as a two state process characterized by one association and one dissociation rate. The Langmuir isotherm itself (Eq. [19]) is a two state model. Hybridization in DNA microarrays is likely to be more complex than a simple two state process. Probes are tethered to the surface by one end and can form a dense brush, which hinders and slows down hybridization. The typical distance between probes is of about 10 nanometers, and the length of a fully stretched 30-mer duplex is of 10 nm and its thickness of 2 nm. Probe sequences in the experiment have also a poly(A) 30-mer spacer (see Table 6). Therefore a single target molecule can interact with more than one probe. Taking this into account, we have extended the two state hybridization model with an additional intermediate state (FIG. 12). Indicating with θ₁ and θ₂, the fraction of partially and fully hybridized probes on the microarray, the kinetics of these reactions is given by

$$\begin{matrix}\frac{d {θ}_{1}}{dt} = {ck}_{1} ( 1 - {θ}_{1} - {θ}_{2} ) + {k}_{- 2} {θ}_{- 2} - ( {k}_{- 1} + {k}_{2} ) {θ}_{1} & [ 20 ] \\ \frac{d {θ}_{2}}{dt} = {k}_{2} {θ}_{2} - {k}_{- 2} {θ}_{2} & [ 21 ]\end{matrix}$$

where c is the target concentration in solution and k₁, k₋₁, k₂ and k₋₂ the four rates involved (see FIG. 12). For simplicity we have assumed that at most a single target molecule can bind to a given probe. The rate constants, using a two state model description, have been measured in several microarray experiments. The hybridization of a common target sequence to a perfect match probe and to a probe containing one mismatch were considered. One is interested in their dependence on ΔG. The following rates were used (at 45° C.):

k₁^{(PM)}=19·10⁻⁴ M⁻¹s⁻¹,

k₁^{(MM)}=21·10⁻⁴ M⁻¹s⁻¹,

k₋₁^{(PM)}=12·10⁴ s⁻¹, and

k₋₁^{(MM)}=29·10⁴ s⁻¹.

While there is more than a factor two of difference in the detachment rates, the attachment rates differ only by 10%. These results are in agreement with observation for kinetic behavior in bulk solution. The probes in the experiment differ by at most by two nucleotides out of 30. It is assumed that both forward rates k₁ and k₂ are sequence independent. The reverse rates are then fixed by the thermodynamics relations

k₋₁=k₁e^{-ΔGT/RT};

k₋₂=k₂e^{-(ΔG-ΔG′)/RT} [22]

where ΔG′ and ΔG are the free energy differences between configurations 1 and 2, and the unbound state, respectively. It is next assumed that ΔG′, the free energy of the partially hybridized state, is monotonically dependent on ΔG. Moreover at unbinding (ΔG=0), also ΔG′ should vanish. As a simple approximation we then take

ΔG′=γΔG(γ<1) [23]

in order to approximate the expected monotonic dependence of ΔG′ from ΔG. The model is then fully characterized by three parameters k₁, k₂ and γ.

FIG. 13 shows a plot of θ₁+θ₂ vs. ΔΔG. These are obtained from the solution of Eqs. [20], [21] for different times and the following choice of parameters k₁=10⁵M⁻¹s⁻¹, k₂=1 s⁻¹ and γ=1/3 (for k₁ the value was used typically measured in kinetic experiments on microarrays, while k₂ and y are chosen to fit experimental data). In FIG. 13 thin solid lines are isotherms at finite times, while the thick line is the equilibrium isotherm (t→×∞).

As time increases the equilibrium intensity is approached from below. To gain some more insight the limit of fast equilibration is considered for Eq. [20]. We then solve Eq. [21] using for θ₁ its equilibrium value:

$${θ}_{1}^{( eq )} = \frac{{ce}^{Δ {G}^{′} / RT}}{c ( {e}^{Δ G / RT} + {e}^{Δ G / RT} ) + 1} \approx {ce}^{Δ {G}^{′} / RT} \qquad [ 24 ]$$

where the low concentration limit ce^{ΔG/RT}«1 was taken. We have then

θ₂(t)=ce^{ΔG/RT}(1-e^{-t/}^{_{τ}}) [25]

τ⁻¹=k₋₂=k₂e^{-(1-γ)ΔG/RT} [26]

which is the inverse relaxation time. To get this Eqs. [22] and [24] were used in the limit ce^{ΔG/RT}«1.

The relaxation time depends on ΔG: weakly bounded sequences (small ΔG) equilibrate faster than strongly bounded ones (large ΔG). For fast equilibrating sequences (τ« t) one recovers from Eq. [25] the usual Langmuir equilibrium; for sequences with long equilibration times τ» t Eq. [25] is expanded to lowest order in t/τ. With this approximation we find that for a given time t

$${θ}_{2} ( t ) = \{ \begin{matrix}{ce}^{Δ G / RT} & Δ G ≪ Δ {G}^{*} \\ {ctk}_{2} {e}^{γΔ G / RT} & Δ G ≫ Δ {G}^{*}\end{matrix} \qquad [ 27 ]$$

where ΔG* is a crossover free energy which depends on time and is obtained by setting τ=t in Eq. [26]. The measured intensity is I=A(θ1+θ2)), however for any realistic choice of free energies θ₁<<θ₂, hence once can approximate I≈Aθ₂. Equation [27] reproduces the two slopes in the log/vs. ΔΔG plots as seen in the experiments. It shows that the non-equilibrium regime is characterized by a slope equal to γ/RT. Turning now to experimental results, routinely, hybridization experiments are performed at constant temperature and buffer conditions for about 15 h. Experiments were performed at shorter and longer hybridization times up to more than 86 h. Once the desired hybridization time has been reached the experiment is stopped, the microarray washed and scanned to measure the emitted fluorescence from every spot. Experiments at different hybridization times thus require different slides.

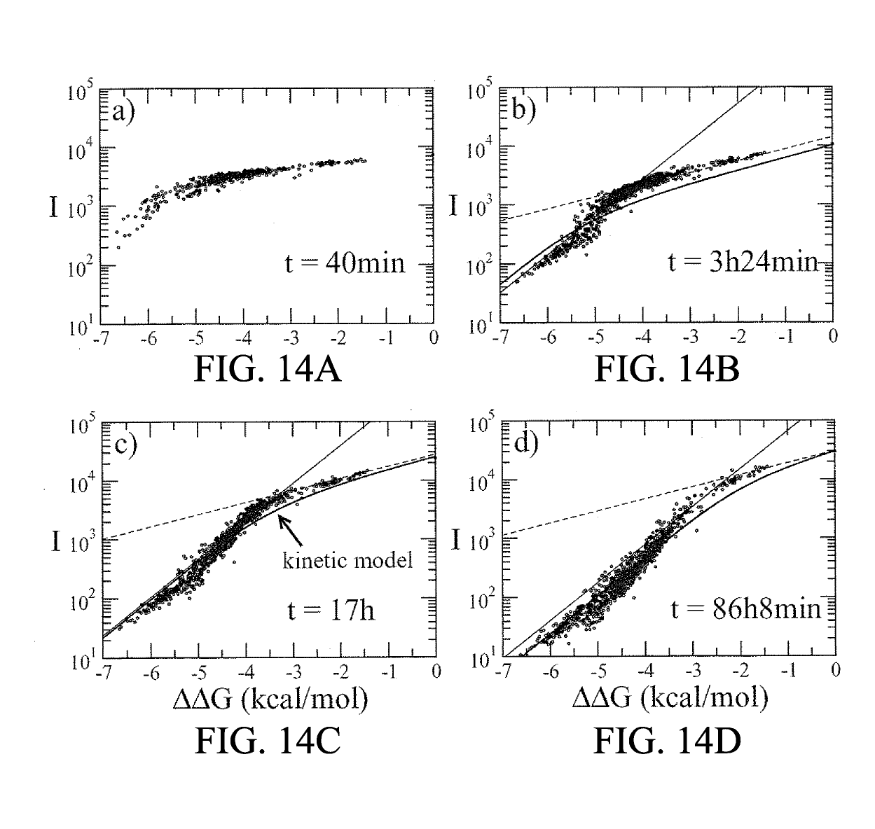

FIGS. 14A-14D shows a plot of/vs. ΔΔG for a 30-mer target at four different times and for a concentration of 50 pM (the 17 h hybridization data are those already shown in FIG. 11). As the hybridization time increases a larger fraction of the data aligns along a line with a slope 1/RT over the full range of intensities, confirming that the observed deviations from the Langmuir model are due to the breakdown of thermodynamic equilibrium. Surprisingly, full equilibrium has not been reached here even after 85 hours. Apart from the shortest hybridization time the data are in agreement with the behavior predicted by Eq. [27]. From the slope of the dashed line, using Eq. [27] it is estimated that γ=0.32. As full hybridization involves a stretch of 30 nucleotides, this suggests that in the partially hybridized state the target and probe are bound for 10 nucleotides, the turn of an helix. The slope of the non-equilibrium regime of FIG. 14A is smaller than that of the dashed lines of FIGS. 14B, 14C and 14D. This is probably due to the protocol followed (this is the standard protocol): at time t=0 both the solution containing the target molecules and the array are at room temperature.

They are then placed into an oven for the duration of the experiment at a constant temperature (T=65° C. for all experiments described in this letter). The slope is reminiscent of an initial “low” temperature hybridization.

When comparing FIGS. 14B, 14C and 14D one notices an overall decrease of the intensity scale, leading to a normalization of the constant A in Eq. [19]. This is probably due to some degradation of the fluorophores (not surprising in view of the time span involved in the experiment). The solid lines in FIGS. 14B, 14C and 14D are plots of the intensity I=A (θ₁+θ₂), where A was adjusted to match the global intensity scale. The agreement with the model is reasonable although not perfect.

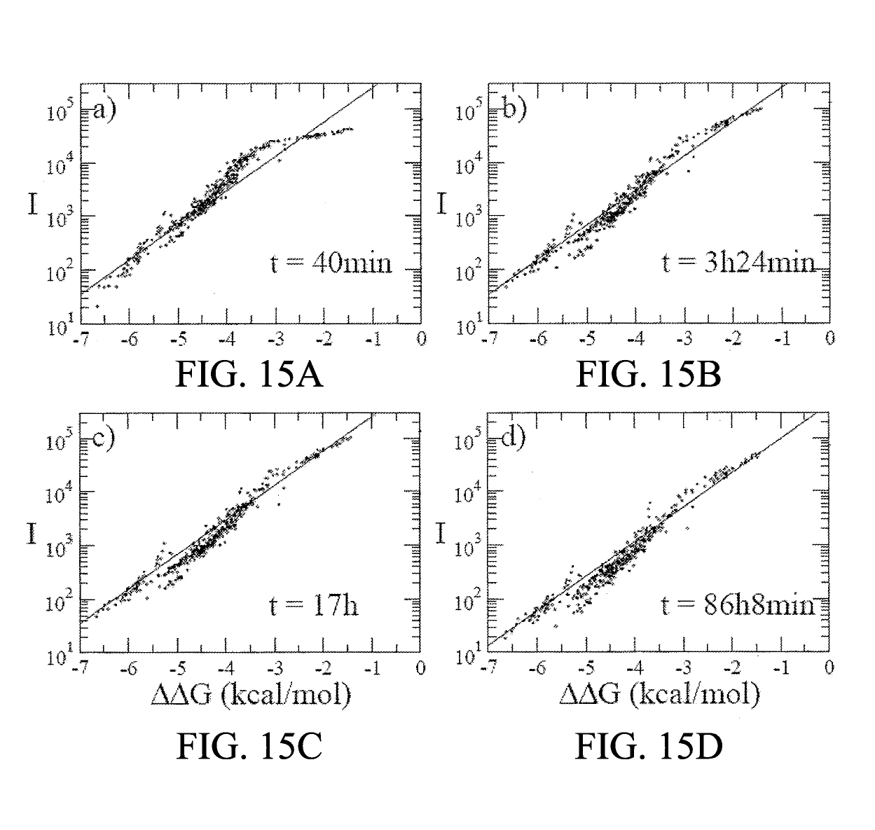

Turning now to the case of hybridization to the shorter target sequence (25-mer, see Table 6). Data are shown in FIGS. 15A-15D. It is expected that faster equilibration for 25-mers occurs because the sequence has a lower ΔG and because for shorter target sequences it is expected that less entanglement occurs hence an increase of the k₂ rate. Both effects lead to smaller τ (see Eq. [26]). As can be seen in FIGS. 15A-15D the only deviation from the equilibrium isotherm are for the shortest hybridization time. The agreement with Eq. [19] is over three orders of magnitude in the intensity scale. Note an overall decrease of the intensity scale as observed in FIGS. 14A-14D. The results are consistent with the idea that interaction of target molecule with multiple probes is responsible to the observed non-equilibrium behavior. The present example shows that hybridization in DNA microarrays under standard conditions is characterized by a relaxation time which may largely exceed the experimental time. Since typical biological experiments involve target strands of 30-50 nucleotides, it is believed that the breakdown of equilibrium shown here on Agilent arrays, may occur in many different microarrays platforms and in biological experiments. It is found that in the non-equilibrium regime the intensities are distributed according to an exponential distribution e^{γΔG/RT}, with γ<1. The breakdown of equilibrium carries important consequences: it lowers the specificity of the microarrays as devices for the detection of a desired sequence from a complex mixture. This can be illustrated with an example. Consider a probe at the microarray surface and two different sequences in solution: one perfect matching with the probe (at concentration c_{PM}) and one with a mismatch (at concentration C_{MM}). In the equilibrium regime the two sequences hybridize to the probe with probabilities proportional to C_{PM}e^{ΔG}^{_{PM/}}^{RT} and c_{MM}e^{ΔG}^{_{MM/}}^{RT}, respectively. Assuming for simplicity equal target concentrations c_{PM}=C_{MM} one has that the ratio of the two contributions is e^{(ΔG}^{_{MM}}^{-ΔG}^{_{PM}})/^{RT}≈0.05 where a typical value ΔG_{MM}-ΔG_{PM}=2 kcal/mol and a temperature of T=65° C. is used. In the non-equilibrium regime, due to the presence of a factor γ=1/3 in the exponential the ratio is about 0.4. Therefore in the non-equilibrium regime a significant fraction of a measured signal may be due to hybridization to non-complementary targets a phenomenon known as cross-hybridization. For an optimal functioning of the microarrays it is then desirable to work under equilibrium conditions. Several parameters may influence the relaxation time as temperature, salt and buffer conditions. The experimental setup discussed provides a good test of equilibrium (single line vs. broken line in a/vs. ΔΔG plot) and can be used to investigate the best working conditions for an optimal hybridization.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The foregoing description details certain embodiments of the invention. It will be appreciated, however, that no matter how detailed the foregoing appears in text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

1. A method for determining in a sample solution the presence of a target nucleic acid of interest, wherein the target nucleic acid of interest differs from one or more other target nucleic acids by only one or two nucleotides, the method comprising the steps of:
(a) providing
a first plurality of different probes wherein each probe comprises a nucleic acid that is perfectly complementary to one nucleic acid of a set of target nucleic acids which differ from the target nucleic acid of interest in only one or two nucleotides; and
a second plurality of different probes, wherein each probe comprises a nucleic acid that is non-perfectly complementary to each of said set of target nucleic acids which differ from the target nucleic acid of interest in only one or two nucleotides,
(b) performing hybridization, said hybridization comprising contacting the sample solution with each probe of said first and second plurality of probes, and detecting a hybridization signal for each probe with the sample solution;
(c) determining the hybridization free energy for the hybridization between each probe of said first and said second plurality of probes and the target nucleic acid of interest, based on a known model for estimating the hybridization free energy as a sum of dinucleotide parameters; and
(d) determining the presence of said target nucleic acid of interest based on the relationship between said hybridization signals detected for said plurality of probes in step (b) and said hybridization free energy determined in step (c).

2. The method of claim 1, wherein said plurality of probes comprises at least 100 probes of a length of between 15 and 50 nucleotides.

3. The method of claim 1 wherein, if in step (d) said target nucleic acid of interest is determined not to be present in said sample solution, then said steps (c) and (d) are repeated based on a another target nucleic acid of interest and said method comprises determining the presence of said other target nucleic acid of interest in said sample.

4. The method according to claim 1 wherein the hybridization experiment of step (b) comprises or measures the hybridization between a target nucleic acid in solution and a probe bound at a surface.

5. The method according to claim 1, wherein the hybridization experiment of step (b) is performed in a microarray wherein each probe is provided to separate microarray spots.

6. The method of claim 1, wherein the sample solution comprises one target nucleic acid differing from the target nucleic acid of interest in one or two non-complementary elements, present in significantly higher concentration than said target nucleic acid of interest.

7. The method of claim 1, wherein said target nucleic acid of interest is a DNA sequence comprising a single nucleotide polymorphism.

8. The method of claim 1, wherein said target nucleic acid of interest is a viral, bacterial, or mammal DNA sequence.

9. The method of claim 1, wherein in step (b) the hybridization signal is induced by emission of a label associated with a hybrid formed by binding of a target nucleic acid and a probe.

10. The method of claim 9, wherein said label is associated with said target nucleic acid in said sample.

11. The method of claim 1, wherein said method further comprises the step of estimating the concentration of the target nucleic acid of interest based on previously performed calibration experiments or by calculation based on an extended equation correlating the logarithm of the hybridization signal intensity with the hybridization free energy.

12. The method of claim 1, wherein at least one step of the method is implemented as a computer program product, configured for performing, when executed on a computing device, at least one step of the method according to claim 1.

13. The method of claim 1, wherein step (d) comprises determining whether said relationship between said hybridization signals measured for said plurality of probes in step (b) and said estimated or determined hybridization free energy in step (c) corresponds to the logarithmic relationship for:
I=A. c. e^{(-ΔG}/_{RT)} or with the linear relationship between log(I) and −ΔG/RT;
wherein I is the hybridization intensity, A is an amplification factor, c the concentration of the target in solution and ΔG=hybridization free energy, T the experimental temperature and R the gas constant; and
wherein deviating from said logarithmic or linear relationship indicates the absence of said target nucleic acid of interest in the sample; and wherein not deviating from said logarithmic or linear relationship indicates the presence and/or concentration of said target nucleic acid of interest in the sample.

14. The method of claim 1, wherein step (d) comprises the steps
(d1) subtracting from all hybridization free energies estimated in step (c) the hybridization free energy between said target nucleic acid of interest and its perfectly matching probe, thus obtaining a set of ΔΔG values;
(d2) comparing the relation of said set of hybridization intensity data as a function of the ΔΔG values with the logarithmic relationship
I=A. c. e^{(-ΔΔG}/_{RT)} or with the linear relationship between log(I) and −ΔΔG/RT for a range of hybridization free energies;
wherein I is the hybridization intensity, A is an amplification factor, c the concentration of the target in solution and ΔG=hybridization free energy, T the experimental temperature and R the gas constant; and
wherein deviating from said logarithmic or linear relationship indicates the absence of said target nucleic acid of interest in said sample; and wherein not deviating from said logarithmic or linear relationship indicates the presence of said target nucleic acid of interest in said sample.
